SEQUENCE LISTING (3 sequences)

SEQ ID NO: 1 - protein, 1265 aa, Homo sapiens
MSTTVNVDSLAEYEKSQIKRALELGTVMTVFSFRKSTPERRTVQVIMETRQVAWSKTADKIEGFLDIMEIKEIRPGKNSKDFERAKAVRQKEDCCFTILYGTQFVLSTLSLAADSKEDAVNWLSGLKILHQEAMNASTPTIIESWLRKQIYSVDQTRRNSISLRELKTILPLINFKVSSAKFLKDKFVEIGAHKDELSFEQFHLFYKKLMFEQQKSILDEFKKDSSVFILGNTDRPDASAVYLHDFQRFLIHEQQEHWAQDLNKVRERMTKFIDDTMRETAEPFLFVDEFLTYLFSRENSIWDEKYDAVDMQDMNNPLSHYWISSSHNTYLTGDQLRSESSPEAYIRCLRMGCRCIELDCWDGPDGKPVIYHGWTRTTKIKFDDVVQAIKDHAFVTSSFPVILSIEEHCSVEQQRHMAKAFKEVFGDLLLTKPTEASADQLPSPSQLREKIIIKHKKLGPRGDVDVNMEDKKDEHKQQGELYMWDSIDQKWTRHYCAIADAKLSFSDDIEQTMEEEVPQDIPPTELHFGEKWFHKKVEKRTSAEKLLQEYCMETGGKDGTFLVRESETFPNDYTLSFWRSGRVQHCRIRSTMEGGTLKYYLTDNLTFSSIYALIQHYRETHLRCAEFELRLTDPVPNPNPHESKPWYYDSLSRGEAEDMLMRIPRDGAFLIRKREGSDSYAITFRARGKVKHCRINRDGRHFVLGTSAYFESLVELVSYYEKHSLYRKMRLRYPVTPELLERYNMERDINSLYDVSRMYVDPSEINPSMPQRTVKALYDYKAKRSDELSFCRGALIHNVSKEPGGWWKGDYGTRIQQYFPSNYVEDISTADFEELEKQIIEDNPLGSLCRGILDLNTYNVVKAPQGKNQKSFVFILEPKQQGDPPVEFATDRVEELFEWFQSIREITWKIDTKENNMKYWEKNQSIAIELSDLVVYCKPTSKTKDNLENPDFREIRSFVETKADSIIRQKPVDLLKYNQKGLTRVYPKGQRVDSSNYDPFRLWLCGSQMVALNFQTADKYMQMNHALFSLNGRTGYVLQPESMRTEKYDPMPPESQRKILMTLTVKVLGARHLPKLGRSIACPFVEVEICGAEYDNNKFKTTVVNDNGLSPIWAPTQEKVTFEIYDPNLAFLRFVVYEEDMFSDPNFLAHATYPIKAVKSGFRSVPLKNGYSEDIELASLLVFCEMRPVLESEEELYSSCRQLRRRQEELNNQLFLYDTHQNLRNANRDALVKEFSVNENQLQLYQEKCNKRLREKRVSNSKFYS

SEQ ID NO: 2 - RNA, 3798 nt, Homo sapiens
  source 1..3798: mol_type = mRNA
atgtccaccacggtcaatgtagattcccttgcggaatatgagaagagccagatcaagagagccctggagctggggacggtgatgactgtgttcagcttccgcaagtccacccccgagcggagaaccgtccaggtgatcatggagacgcggcaggtggcctggagcaagaccgctgacaagatcgagggcttcttggatatcatggaaataaaagaaatccgcccagggaagaactccaaagatttcgagcgagcaaaagcagttcgccagaaagaagactgctgcttcaccatcctatatggcactcagttcgtcctcagcacgctcagcttggcagctgactctaaagaggatgcagttaactggctctctggcttgaaaatcttacaccaggaagcgatgaatgcgtccacgcccaccattatcgagagttggctgagaaagcagatatattctgtggatcaaaccagaagaaacagcatcagtctccgagagttgaagaccatcttgcccctgatcaactttaaagtgagcagtgccaagttccttaaagataagtttgtggaaataggagcacacaaagatgagctcagctttgaacagttccatctcttctataaaaaacttatgtttgaacagcaaaaatcgattctcgatgaattcaaaaaggattcgtccgtgttcatcctggggaacactgacaggccggatgcctctgctgtttacctgcatgacttccagaggtttctcatacatgaacagcaggagcattgggctcaggatctgaacaaagtccgtgagcggatgacaaagttcattgatgacaccatgcgtgaaactgctgagcctttcttgtttgtggatgagttcctcacgtacctgttttcacgagaaaacagcatctgggatgagaagtatgacgcggtggacatgcaggacatgaacaaccccctgtctcattactggatctcctcgtcacataacacgtaccttacaggtgaccagctgcggagcgagtcgtccccagaagcttacatccgctgcctgcgcatgggctgtcgctgcattgaactggactgctgggacgggcccgatgggaagccggtcatctaccatggctggacgcggactaccaagatcaagtttgacgacgtcgtgcaggccatcaaagaccacgcctttgttacctcgagcttcccagtgatcctgtccatcgaggagcactgcagcgtggagcaacagcgtcacatggccaaggccttcaaggaagtatttggcgacctgctgttgacgaagcccacggaggccagtgctgaccagctgccctcgcccagccagctgcgggagaagatcatcatcaagcataagaagctgggcccccgaggcgatgtggatgtcaacatggaggacaagaaggacgaacacaagcaacagggggagctgtacatgtgggattccattgaccagaaatggactcggcactactgcgccattgccgatgccaagctgtccttcagtgatgacattgaacagactatggaggaggaagtgccccaggatataccccctacagaactacattttggggagaaatggttccacaagaaggtggagaagaggacgagtgccgagaagttgctgcaggaatactgcatggagacggggggcaaggatggcaccttcctggttcgggagagcgagaccttccccaatgactacaccctgtccttctggcggtcaggccgggtccagcactgccggatccgctccaccatggagggcgggaccctgaaatactacttgactgacaacctcaccttcagcagcatctatgccctcatccagcactaccgcgagacgcacctgcgctgcgccgagttcgagctgcggctcacggaccctgtgcccaaccccaacccccacgagtccaagccgtggtactatgacagcctgagccgcggagaggcagaggacatgctgatgaggattccccgggacggggccttcctgatccggaagcgagaggggagcgactcctatgccatcaccttcagggctaggggcaaggtaaagcattgtcgcatcaaccgggacggccggcactttgtgctggggacctccgcctattttgagagtctggtggagctcgtcagttactacgagaagcattcactctaccgaaagatgagactgcgctaccccgtgacccccgagctcctggagcgctacaatatggaaagagatataaactccctctacgacgtcagcagaatgtatgtggatcccagtgaaatcaatccgtccatgcctcagagaaccgtgaaagctctgtatgactacaaagccaagcgaagcgatgagctgagcttctgccgtggtgccctcatccacaatgtctccaaggagcccgggggctggtggaaaggagactatggaaccaggatccagcagtacttcccatccaactacgtcgaggacatctcaactgcagacttcgaggagctagaaaagcagattattgaagacaatcccttagggtctctttgcagaggaatattggacctcaatacctataacgtcgtgaaagcccctcagggaaaaaaccagaagtcctttgtcttcatcctggagcccaagcagcagggcgatcctccggtggagtttgccacagacagggtggaggagctctttgagtggtttcagagcatccgagagatcacctggaagattgacaccaaggagaacaacatgaagtactgggagaagaaccagtccatcgccatcgagctctctgacctggttgtctactgcaaaccaaccagcaaaaccaaggacaacttagaaaatcctgacttccgagaaatccgctcctttgtggagacgaaggctgacagcatcatcagacagaagcccgtcgacctcctgaagtacaatcaaaagggcctgacccgcgtctacccaaagggacaaagagttgactcttcaaactacgaccccttccgcctctggctgtgcggttctcagatggtggcactcaatttccagacggcagataagtacatgcagatgaatcacgcattgttttctctcaatgggcgcacgggctacgttctgcagcctgagagcatgaggacagagaaatatgacccgatgccacccgagtcccagaggaagatcctgatgacgctgacagtcaaggttctcggtgctcgccatctccccaaacttggacgaagtattgcctgtccctttgtagaagtggagatctgtggagccgagtatgacaacaacaagttcaagacgacggttgtgaatgataatggcctcagccctatctgggctccaacacaggagaaggtgacatttgaaatttatgacccaaacctggcatttctgcgctttgtggtttatgaagaagatatgttcagcgatcccaactttcttgctcatgccacttaccccattaaagcagtcaaatcaggattcaggtccgttcctctgaagaatgggtacagcgaggacatagagctggcttccctcctggttttctgtgagatgcggccagtcctggagagcgaagaggaactttactcctcctgtcgccagctgaggaggcggcaagaagaactgaacaaccagctctttctgtatgacacacaccagaacttgcgcaatgccaaccgggatgccctggttaaagagttcagtgttaatgagaaccagctccagctgtaccaggagaaatgcaacaagaggttaagagagaagagagtcagcaacagcaagttttactcatag

SEQ ID NO: 3 - RNA, 2575 nt, Homo sapiens
  source 1..2575: mol_type = mRNA
agactgtccttcctctctggactgtaagaatatgtctccagggccagtgtctgctgcgatcgagtcccaccttccaagtcctggcatctcaatgcatctgggaagctacctgcattaagtcaggactgagcacacaggtgaactccagaaagaagaagctatggccgcagtgattctggagagcatctttctgaagcgatcccaacagaaaaagaaaacatcacctctaaacttcaagaagcgcctgtttctcttgaccgtgcacaaactctcctactatgagtatgactttgaacgtgggagaagaggcagtaagaagggttcaatagatgttgagaagatcacttgtgttgaaacagtggttcctgaaaaaaatcctcctccagaaagacagattccgagaagaggtgaagagtccagtgaaatggagcaaatttcaatcattgaaaggttcccttatcccttccaggttgtatatgatgaagggcctctctacgtcttctccccaactgaagaactaaggaagcggtggattcaccagctcaaaaacgtaatccggtacaacagtgatctggttcagaaatatcacccttgcttctggatcgatgggcagtatctctgctgctctcagacagccaaaaatgctatgggctgccaaattttggagaacaggaatggaagcttaaaacctgggagttctcaccggaagacaaaaaagcctcttcccccaacgcctgaggaggaccagatcttgaaaaagccactaccgcctgagccagcagcagcaccagtctccacaagtgagctgaaaaaggttgtggccctttatgattacatgccaatgaatgcaaatgatctacagctgcggaagggtgatgaatattttatcttggaggaaagcaacttaccatggtggagagcacgagataaaaatgggcaggaaggctacattcctagtaactatgtcactgaagcagaagactccatagaaatgtatgagtggtattccaaacacatgactcggagtcaggctgagcaactgctaaagcaagaggggaaagaaggaggtttcattgtcagagactccagcaaagctggcaaatatacagtgtctgtgtttgctaaatccacaggggaccctcaaggggtgatacgtcattatgttgtgtgttccacacctcagagccagtattacctggctgagaagcaccttttcagcaccatccctgagctcattaactaccatcagcacaactctgcaggactcatatccaggctcaaatatccagtgtctcaacaaaacaagaatgcaccttccactgcaggcctgggatacggatcatgggaaattgatccaaaggacctgaccttcttgaaggagctggggactggacaatttggggtagtgaagtatgggaaatggagaggccagtacgacgtggccatcaagatgatcaaagaaggctccatgtctgaagatgaattcattgaagaagccaaagtcatgatgaatctttcccatgagaagctggtgcagttgtatggcgtctgcaccaagcagcgccccatcttcatcatcactgagtacatggccaatggctgcctcctgaactacctgagggagatgcgccaccgcttccagactcagcagctgctagagatgtgcaaggatgtctgtgaagccatggaatacctggagtcaaagcagttccttcaccgagacctggcagctcgaaactgtttggtaaacgatcaaggagttgttaaagtatctgatttcggcctgtccaggtatgtcctggatgatgaatacacaagctcagtaggctccaaatttccagtccggtggtccccaccggaagtcctgatgtatagcaagttcagcagcaaatctgacatttgggcttttggggttttgatgtgggaaatttactccctggggaagatgccatatgagagatttactaacagtgagactgctgaacacattgcccaaggcctacgtctctacaggcctcatctggcttcagagaaggtatataccatcatgtacagttgctggcatgagaaagcagatgagcgtcccactttcaaaattcttctgagcaatattctagatgtcatggatgaagaatcctgagctcgccaataagcttcttggttctacttctcttctccacaagccccaatttcactttctcagaggaaatcccaagcttaggagccctggagcctttgtgctcccactcaatacaaaaaggcccctctctacatctgggaatgcacctcttctttgattccctgggatagtggcttctgagcaaaggccaagaaattattgtgcctgaaatttcccgagagaattaagacagactgaatttgcgatgaaaatattttttaggagggaggatgtaaatagccgcacaaaggggtccaacagctctttgagtaggcatttggtagagcttgggggtgtgtgtgtgggggtggaccgaatttggcaagaatgaaatggtgtcataaagatgggaggggagggtgttttgataaaataaaattactagaaagcttgaaa

(19) United States
(12) Patent Application Publication
(10) Pub. No.: US 2025/0352548 A1
(43) Pub. Date: Nov. 20, 2025

(54) BTK Reducing Molecules for Treatment of Cancers and Immune System Disorders

(71) Applicant: Accutar Biotechnology, Inc., Cranbury, NJ (US)

(72) Inventors: Wei He, Zionsville, IN (US); Yimin Qian, Plainsboro, NJ (US); Feng Chen, Livingston, NJ (US); Jie Su, New York, NY (US); Jie Fan, New York, NY (US)

(21) Appl. No.: 18/869,693

(22) PCT Filed: May 30, 2023
(86) PCT No.: PCT/US23/67596

§ 371 Date: Nov. 26, 2024

Related U.S. Application Data
(60) Provisional application No. 63/365,509, filed on May 31, 2022.

(51) Int. Cl. A61K 31/519; A61K 31/4985; A61K 31/7105; A61P 35/02; C12Q 1/6886

(52) U.S. Cl. CPC A61K 31/519; A61K 31/4985; A61K 31/7105; A61P 35/02; C12Q 1/6886; C12Q 2600/156

(57) ABSTRACT

The present disclosure is generally directed to methods of treating a disease or disorder associated with dysfunctional phosphatidylinositol-specific phospholipase Cγ2 (PLCγ2), such as cancers and immune system disorders, using Bruton's tyrosine kinase (BTK) reducing molecules, such as BTK degrader molecules.

### CROSS-REFERENCE TO RELATED APPLICATIONS

This application claims priority to U.S. Provisional Application No. 63/365,509 filed 31 May 2022, the entire contents of which are hereby incorporated by reference in their entirety.

### SEQUENCE LISTING

The Sequence Listing associated with this application is filed in electronic format as a XML file and hereby incorporated by reference into the specification in its entirety. The name of the XML file containing the Sequence Listing is 0437_0002_PCT_SL.xml and the size of the text file is 11 KB.

### FIELD OF THE DISCLOSURE

The present disclosure is generally directed to methods of treating a disease or disorder associated with dysfunctional phosphatidylinositol-specific phospholipase Cγ2 (PLCγ2), such as cancers and immune system disorders, using Bruton's tyrosine kinase (BTK) reducing molecules, such as BTK degrader molecules.

### BACKGROUND OF THE DISCLOSURE

The B-cell receptor signaling pathway is important for proper B-cell development, activation, proliferation, differentiation and consequently for adaptive immune responses. Phosphatidylinositol-specific phospholipase Cγ2 (PLCγ2) is a signaling enzyme activated by a variety of cell surface receptors including B cell receptors. These receptors recruit kinases, such as tyrosine-protein kinases SYK and LYN, Bruton's tyrosine kinase (BTK), and B-cell linker protein (BLNK), to phosphorylate and activate PLCγ2, which then generates important second messenger molecules, inositol 1,4,5-trisphosphate (IP3) and diacylglycerol (DAG). IP3 and DAG subsequently mediate diverse biological functions, including cellular proliferation, endocytosis, and calcium flux.

While the interaction and interdependence between the various components of the B cell receptor pathway is not fully understood, it is widely accepted that signaling through the B cell receptor pathway relies on activated BTK followed by the transduction of signals to downstream effectors via PLCγ2. Therefore, gain-of-function mutations in downstream PLCγ2 are considered one of the major acquired resistance mechanisms to BTK inhibitors in B-cell lymphoma, such as chronic lymphocytic leukemia (CLL). See e.g., Woyach et al., J. Clinical Oncology, 2017, 35(13):1437-1443; Ahn et al., Blood, 2017, 129(11):1469-1479.

PLCγ2 dysfunction is also associated with a variety of diseases, including those with an immunological basis such as inflammation, autoimmunity, immunodeficiency, and allergy, as well as hematological malignancies. See e.g., Jackson et al., J. Biol. Chem., 2021, 297(2):100905. Thus, manipulation of PLCγ2 activity could be considered as a therapeutic modality in some malignancies and immune disorders.

Accordingly, there remains a need for new medications to treat diseases or disorders associated with PLCγ2 dysfunction.

### SUMMARY OF THE DISCLOSURE

This application demonstrates that reduction or elimination of BTK by, for example, proteolytically degrading BTK or reducing BTK expression—in contrast to simply inhibiting BTK's catalytic function—can be used to target and kill cells having constitutively activated PLCγ2. Given the downstream location of PLCγ2 relative to BTK in the signaling cascade and clinical observation of gain-of-function mutations in PLCγ2 resulting in acquired resistance to BTK inhibitors, it is unexpected that a BTK reducing molecule can be used to treat diseases or disorders associated with constitutively active PLCγ2.

Disclosed herein are methods of treating diseases or disorders associated with constitutively activated PLCγ2, such as cancers and immune system disorders, comprising administering to a subject in need thereof an effective amount of a Bruton's tyrosine kinase (BTK) reducing molecule. In some embodiments, prior to administering to the subject the BTK reducing molecule, the subject has been identified as having constitutively activated PLCγ2 in one or more cells. In some embodiments, the methods further comprise identifying a cell obtained from the subject as having the constitutively activated PLCγ2 relative to a control cell or identifying a cell obtained from the subject as having one or more gain-of-function mutations in a gene encoding PLCγ2. In some embodiments, the subject is a human.

In some embodiments, the constitutively activated PLCγ2 in the subject is caused by one or more gain-of-function mutations in a gene encoding PLCγ2, such as a deletion of one or more nucleotides in the gene encoding PLCγ2 or one or more mutations causing a substitution at P139, T168, I169, D334, Y482, N571, P664, R665, S707, A708, S718, R742, L845, L848, D993, D1140, M1141, F1142, or D1144 of PLCγ2 (SEQ ID NO: 1). In some embodiments, the one or more gain-of-function mutations comprise one or more of the following mutations: P139S, T168A, I169V, D334H, Y482H, N571S, P664S, R665W, S707Y, S707P, S707F, A708P, S718R, R742P, L845F, L845V, L848P, D993Y, D993H, D1140G, D1140Y, D1140N, D1140E, D1140V, M1141L, M1141R, M1141K, F1142L, D1144N, or D1144G of SEQ ID NO: 1, or a deletion of at least amino acids L845-L848, or a deletion of at least amino acids S707-A708 of SEQ ID NO: 1, or a deletion of one or more nucleotides in exons 19-22 of the gene encoding PLCγ2. In some embodiments, the one or more gain-of-function mutations are located within a regulatory domain and/or a calcium binding domain of PLCγ2, such as mutations at amino acids Y482, N571, P664, R665, S707, A708, S718, R742, L845, L848, D1140, M1141, F1142, or D1144 of SEQ ID NO: 1.

In some embodiments, the disease or disorder associated with the constitutively activated PLCγ2 is a cancer, such as a solid tumor or a hematological cancer (e.g., B-cell malignancy, including but not limited to non-Hodgkin lymphoma (NHL), such as chronic lymphocytic leukemia (CLL), small lymphocytic lymphoma (SLL), mantle cell lymphoma (MCL), marginal zone lymphoma (MZL), or Waldenstrom macroglobulinemia (WM)). In some embodiments, the cancer is resistant to a BTK inhibitor, such as ibrutinib, acalabrutinib, zanubrutinib, or tirabrutinib.

In some embodiments, the disease or disorder associated with the constitutively activated PLCγ2 is an immune system disorder, such as PLCγ2-associated antibody deficiency and immune dysregulation syndrome (PLAID), familial cold autoinflammatory syndrome (FCAS3), autoinflammation, antibody deficiency, and immune dysregulation syndrome (APLAID), or common variable immunodeficiency (CVID).

In some embodiments, the BTK reducing molecule is a BTK degrader molecule, such as a compound of Formula I (e.g., Formulae IA-IP) disclosed herein. In some embodiments, the BTK reducing molecule is a nucleic acid inhibitor molecule, such as an antisense oligonucleotide, a microRNA, a RNAi molecule, an antagomir, an aptamer, or a ribozyme.

### BRIEF DESCRIPTION OF THE DRAWINGS

The accompanying drawings, which are incorporated in and constitute a part of this specification, illustrate certain embodiments, and together with the written description, serve to explain certain principles of the methods and devices disclosed herein.

FIG. 1A-1C depict the effect of Ibrutinib, a conventional small molecule BTK inhibitor, and the BTK degrader of Formula I-D of the present disclosure in inhibiting cancer cell growth in vitro 6 days after administration. FIG. 1A: parental REC-1 cells; FIG. 1B: engineered REC-1 cells with PLCγ2 S707Y knock-in mutation; FIG. 1C: engineered REC-1 cells with PLCγ2 L845F knock-in mutation.

FIG. 2A-2C depict the effect of Ibrutinib and the BTK degrader of Formula I-D of the present disclosure in inhibiting tumor growth in vivo 14 days after administration. FIG. 2A: REC-1 mice model; FIG. 2B: REC-1 PLCγ2^{S707Y} knock-in mice model; FIG. 2C: REC-1 PLCγ2^{L845F} knock-in mice model.

### DETAILED DESCRIPTION THE DISCLOSURE

Reference will now be made in detail to various exemplary embodiments, examples of which are illustrated in the accompanying drawings. It is to be understood that the following detailed description is provided to give the reader a fuller understanding of certain embodiments, features, and details of aspects of the disclosure, and should not be interpreted as a limitation of the scope of the disclosure.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, 1%, ±0.9%, 0.8%, ±0.7%, ±0.6%, ±0.5%, ±0.4%, ±0.3%, ±0.2% or ±0.1% from the specified value as such variations are appropriate to perform the disclosed methods and/or to make and use the disclosed devices. When “about” is present before a series of numbers or a range, it is understood that “about” can modify each of the numbers in the series or range.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

The terms “disease,” “disorder,” and “condition” are used interchangeably herein.

As used herein, the term “in some embodiments” refers to embodiments of all aspects of the disclosure, unless the context clearly indicates otherwise.

As used herein, the term “BTK reducing molecule” refers to a molecule that reduces or eliminates the amount of BTK in contrast to a molecule that might inhibit BTK function (e.g., BTK inhibitor that inhibits kinase activity) but not reduce the amount of BTK. Reducing or eliminating the amount of BTK reduces or eliminates not only BTK's kinase activity, but also reduces or eliminates the ability of BTK to interact with other molecules, include other molecules in the signaling cascade, such as PLCγ2.

A “BTK degrader molecule,” as used herein and defined above, refers to a molecule that reduces or eliminates the amount of BTK by inducing proteolytic degradation of BTK. In some embodiments, the BTK degrader molecule of the disclosure is a proteolysis-targeting chimera.

A “BTK inhibitor,” as used herein, refers to a molecule that blocks BTK's catalytic function, for example, by binding to the catalytic region of the kinase. Because a BTK inhibitor only blocks BTK's catalytic function but otherwise does not reduce or eliminate the amount of BTK in the cells, a “BTK inhibitor,” as used herein, is not a “BTK reducing molecule” or “BTK degrader molecule” as defined herein. Examples of BTK inhibitors include, but are not limited to, ibrutinib, acalabrutinib, zanubrutinib, and tirabrutinib.

As used herein, the “effective amount” of a compound refers to an amount sufficient to elicit the desired biological response. As will be appreciated by those of ordinary skill in this art, the effective amount of a compound or molecule of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment.

The term “gain-of-function mutation,” as used herein, refers to any mutation in a gene in which the protein encoded by said gene (i.e., the mutant protein) acquires a function not normally associated with the normal, wild-type protein. The gain-of-function mutation can be a deletion, addition, or substitution of a nucleotide or nucleotides in the gene which gives rise to the change in the function of the encoded protein. In some embodiments, the gain-of-function mutation changes the function of the mutant protein or causes interactions with other proteins. In other embodiments, the gain-of-function mutation causes a decrease in or removal of normal wild-type protein, for example, by interaction of the altered, mutant protein with said normal, wild-type protein. In the context of the present disclosure, in some embodiments, PLCγ2 mutants that show higher PLCγ2 activity than a normal, wild-type PLCγ2 are considered as gain-of-function mutants and can be identified as gain-of-function mutants using an assay suitable for detecting PLCγ2 activity, such as an assay for analyzing inositol phosphate formation in COS-7 cells transfected with wild-type or mutant PLCγ2 as described in Everett et al. (J. Biol. Chem., 2009, 284(34):23083-23093) or assays for determining intracellular calcium flux related to PLCγ2 function as described in Woyach et al. (N. Engl. J. Med., 2014, 370:2286-2294) and Novice et al. (J. Clin. Immunology, 2020, 40:267-276), all incorporated by reference in their entireties herein. A gain-of-function mutation in PLCγ2 typically leads to higher levels of phospholipase activity as compared to a suitable control cell as measured using a suitable cellular assay, such as that described in Novice et al. (J. Clin. Immunology, 2020, 40:267-276). In some instances, however, as has been observed in certain, non-malignant immune cells, a gain-of-function mutation in PLCγ2 that leads to constitutively activated PLCγ2 may result in decreased PLCγ2-dependent signaling and function. This loss of PLCγ2 downstream function in certain immune cells may be a direct result of chronic signaling caused by a gain-of-function mutation in PLCγ2, similar to how chronic B-cell-receptor stimulation results in calcium currents of diminished amplitude, alteration of signaling cascades, and ultimately, proliferative anergy (Ombrello et al., N. Engl. J. Med., 2012, 366:330-338). In these instances where the gain-of-function mutation in PLCγ2 results in decreased PLCγ2-dependent signaling and function, however, it is still possible to identify the PLCγ2 mutant as a gain-of-function mutation if it shows higher PLCγ2 activity than a normal, wild-type PLCγ2, in an assay for analyzing inositol phosphate formation in COS-7 cells transfected with wild-type or mutant PLCγ2 as described in Everett et al. (J. Biol. Chem., 2009, 284(34):23083-23093) or DT40 cells stably expressing wild-type or mutant PLCγ2 as described in Woyach et al. (N. Engl. J. Med., 2014, 370:2286-2294).

The term “identity” or “identical,” as known in the art, is a relationship between two or more polypeptide sequences or two or more polynucleotide sequences, as determined by comparing the sequences. In the art, “identity” or “identical” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). Typical methods to determine identity are designed to give the largest match between the sequences tested. Methods to determine identity and similarity are codified in publicly available computer programs. Typical computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

As used herein, “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

As used herein, the term “target nucleic acid sequence,” “targeting region,” “target gene,” and the like are used interchangeably and refer to a RNA or DNA sequence that is “targeted,” e.g., for cleavage mediated by a nucleic acid inhibitor molecule that contains a nucleic acid sequence that is partially, substantially, or fully or sufficiently complementary to that target sequence.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

The embodiments disclosed herein are not intended to be limited in any manner by the above exemplary listing of chemical groups and substituents. Those skilled in the art will recognize that several embodiments are possible within the scope and spirit of the present disclosure. The following description illustrates the disclosure and, of course, should not be construed in any way as limiting the scope of the inventions described herein

### Methods of Treatment

Provided herein are methods for treating diseases or disorders associated with constitutively activated PLCγ2 by administering to a subject in need thereof an effective amount of a BTK reducing molecule. PLCγ2 plays important roles in both adaptive and innate immune systems. Particularly in the adaptive immune system, PLCγ2 plays a fundamental role in B cell development, affecting the survival of mature B cells and antibody production, as well as being an integral and immediately proximal component of the B cell receptor (BCR) signaling cascade that occurs when a BCR is bound and stimulated by its cognate antigen. Aside from BCR engagement, there are alternative signaling pathways such as CD38, CD40, IL-4R initiated through extracellular immunological receptors that also rely on PLCγ2 to transduce signals.

This application demonstrates that reduction or elimination of BTK by, for example, proteolytically degrading BTK or reducing BTK expression—in contrast to simply inhibiting BTK's catalytic function—is synthetically lethal to cells having constitutively activated PLCγ2. It is unexpected that a BTK reducing molecule can be used to treat disorders with constitutively active PLCγ2, particularly when gain-of-function mutations in PLCγ2 are considered as one of the major acquired resistance mechanisms to therapeutic BTK inhibitors in B-cell lymphoma. See e.g., Woyach et al., J. Clinical Oncology, 2017, 35(13):1437-1443; Ahn et al., Blood, 2017, 129(11):1469-1479. Given the downstream location of PLCγ2 relative to BTK in the signaling cascade and clinical observation of gain-of-function mutations in PLCγ2 resulting in acquired resistance to BTK inhibitors, one of skill in the art would not expect that reducing or eliminating the amount of BTK in a cell would have any effect on constitutively active PLCγ2. Without intending to be bound by any theory, it appears that reducing or eliminating the amount of BTK not only reduces or eliminates BTK's kinase activity, but also reduces or eliminates BTK-mediated scaffolding interactions with other molecules in the signaling cascade, such as PLCγ2.

Because PLCγ2 dysfunction has been associated with a variety of diseases including those with an immunological basis such as inflammation, autoimmunity, immunodeficiency, and allergy, as well as in hematological malignancies (Jackson et al., J. Biol. Chem., 2021, 297(2):100905), this finding has important implications in the treatment of diseases or disorders associated with constitutively activated PLCγ2, such as cancers and immune system disorders.

Accordingly, in one aspect, the disclosure provides use of BTK reducing molecules for treating a disease or disorder associated with constitutively activated PLCγ2 in a subject in need thereof.

In another aspect, the disclosure provides use of BTK reducing molecules in the manufacture of a medicament for the treatment of a disease or disorder associated with constitutively activated PLCγ2 in a subject in need thereof.

In a further aspect, the disclosure provides a method of treating a disease or disorder associated with constitutively activated PLCγ2, the method comprising administering to a subject in need thereof an effective amount of a BTK reducing molecule. In some embodiments, the subject does not have a mutation in the BTK gene.

In some embodiments, the disease or disorder associated with constitutively activated PLCγ2 is a cancer, such as a hematological cancer or a solid tumor. In some embodiments, the cancer is resistant to a BTK inhibitor, such as is ibrutinib, acalabrutinib, zanubrutinib, or tirabrutinib. In some embodiments, the cancer is ibrutinib-resistant. In some embodiments, the disease or disorder associated with constitutively activated PLCγ2 is an immune system disorder.

Hematological cancers, such as B-cell malignancy, may include, but are not limited to, leukemia, lymphoma, B-cell lymphoma, non-Hodgkin lymphoma (NHL), chronic lymphocyte leukemia (CLL), small lymphocytic lymphoma (SLL), mantle cell lymphoma (MCL), marginal zone lymphoma (MZL), Waldenström's macroglobulinemia (WM), transformed CLL or Richter's transformation, diffuse large B cell lymphoma (DLBCL), follicular lymphoma (FL), central nervous system (CNS) lymphoma, endemic Burkitt lymphoma (EBL), and mucosa-associated lymphoid tissue (MALT)-associated gastric lymphoma, Hodgkin lymphoma (HL), and multiple myeloma (MM). Accordingly, in some embodiments, the disease or disorder associated with constitutively activated PLCγ2 according to the disclosure is B-cell malignancy. In some embodiments, the disease or disorder associated with constitutively activated PLCγ2 according to the disclosure is leukemia, lymphoma, B-cell lymphoma, NHL, CLL, SLL, MCL, MZL, WM, transformed CLL or Richter's transformation, DLBCL, FL, CNS lymphoma, EBL, MALT-associated gastric lymphoma, HL, or MM. In some embodiments, the disease or disorder associated with constitutively active PLCγ2 according to the disclosure is CLL, SLL, MCL, MZL, or WM.

Solid tumors may include, but are not limited to, skin cancer, bladder cancer, breast cancer, uterine cancer, ovarian cancer, urothelial cancer, prostate cancer, lung cancer, colorectal cancer, cervical cancer, liver cancer, head and neck cancer, pancreatic cancer, renal cancer, stomach cancer, cerebral cancer, sarcomas, osteosarcoma, esophageal squamous cell carcinoma, esophageal adenocarcinoma, and mesothelioma. Accordingly, in some embodiments, the disease or disorder associated with constitutively activated PLCγ2 according to the disclosure is skin cancer, bladder cancer, breast cancer, uterine cancer, ovarian cancer, urothelial cancer, prostate cancer, lung cancer, colorectal cancer, cervical cancer, liver cancer, head and neck cancer, pancreatic cancer, renal cancer, stomach cancer, cerebral cancer, sarcomas, osteosarcoma, esophageal squamous cell carcinoma, esophageal adenocarcinoma, or mesothelioma. In some embodiments, the disease or disorder associated with constitutively activated PLCγ2 according to the disclosure is lung cancer, breast cancer, prostate cancer, colorectal cancer, urothelial cancer, pancreatic cancer, or liver cancer.

Immune system disorders may include, but are not limited to, allergy, autoinflammatory diseases, chronic inflammatory disorders, autoimmune diseases, rheumatoid arthritis (RA), osteoarthritis (OA), systemic lupus erythematosus (SLE), multiple sclerosis (MS), Sjogren's syndrome, systemic sclerosis, pemphigus, immune thrombocytopenic purpura (ITP), idiopathic pulmonary fibrosis (IPF), myositis, atopic dermatitis (AD), psoriasis, chronic graft-versus-host disease (GvHD), atherosclerosis, asthma, chronic obstructive pulmonary disease (COPD), and inflammatory bowel disease (IBD). Immune system disorders caused by PLCγ2 dysfunction, particularly constitutively activated PLCγ2, may include, but are not limited to, PLCγ2-associated antibody deficiency and immune dysregulation syndrome (PLAID), familial cold autoinflammatory syndrome 3 (FCAS3), autoinflammation, antibody deficiency, and immune dysregulation syndrome (APLAID), common variable immunodeficiency (CVID). Accordingly, in some embodiments, the disease or disorder associated with constitutively activated PLCγ2 according to the disclosure is allergy, autoinflammatory diseases, chronic inflammatory disorders, autoimmune diseases, autoinflammatory diseases, chronic inflammatory disorders, RA, OA, SLE, MS, Sjogren's syndrome, systemic sclerosis, ITP, IPF, AD, psoriasis, chronic GvHD, atherosclerosis, asthma, COPD, or IBD. In some embodiments, the disease or disorder associated with constitutively activated PLCγ2 according to the disclosure is PLAID, FCAS3, APLAID, or CVID.

In some embodiments, prior to administering to the subject the BTK reducing molecule, the subject has been identified as having constitutively activated PLCγ2 in one or more cells. Accordingly, in some embodiments, the methods of the disclosure further comprise identifying a cancer cell or an immune cell obtained from the subject as having the constitutively activated PLCγ2 relative to a control cell. Because constitutively activated PLCγ2 in a cancer cell is due to somatic mutations, the control cell used in identifying whether a cancer cell has constitutively activated PLCγ2 can be a non-cancer cell obtained from the same subject. Conversely, because constitutively activated PLCγ2 in an immune cell is due to germline mutations, the control cell used in identifying whether an immune cell has constitutively activated PLCγ2 can be a cell obtained from a healthy subject. A “healthy subject,” as used herein, refers to a subject who does not have, or is not suspected of having, any disease or disorder, particularly an immune system disorder. Preferably, the cell obtained from the healthy subject is from the same tissue as the immune cell obtained from the subject.

In some embodiments therefore, provided herein is a method for treating a cancer associated with a constitutively activated PLCγ2 in a subject in need thereof, comprising identifying a cancer cell obtained from the subject as having the constitutively activated PLCγ2 relative to a non-cancer cell obtained from the same subject and administering to the subject an effective amount of a BTK reducing molecule. In some embodiments, provided herein is a method for treating an immune system disorder associated with a constitutively activated PLCγ2 in a subject in need thereof, comprising identifying an immune cell obtained from the subject as having the constitutively activated PLCγ2 relative to a cell obtained from a healthy subject and administering to the subject an effective amount of a BTK reducing molecule.

Whether a cell, such as a cancer cell or immune cell, has constitutively activated PLCγ2 can be determined by any method known in the art. For instance, because PLCγ2 catalyzes the hydrolysis of the cell membrane lipid phosphatidylinositol 4,5-bisphosphate (PIP₂) to generate inositol 3,4,5-trisphosphate (IP₃) and diacylglycerol (DAG), the activity of PLCγ2 may be determined by measuring the production of IP₃ through the hydrolysis of substrate PIP₂ in a cell. Cells having constitutively activated PLCγ2 will have increased ability to produce IP₃ as compared to control cells, which can be determined using standard chemical or radiolabeling techniques known in the art. For instance, measurement of IP₃ kinetics can be performed based upon isotope labeling of cell populations followed by extraction and HPLC-separation of the active (1,4,5) isomer from the inactive (1,3,4) one (Balla et al., PNAS, 1986, 83(24):9323-9327). Alternatively, radio-receptor assays can also be used to measure absolute mass changes of IP₃ from populations of cells (Matsu-ura et al., Journal of Cell Biology, 2006, 173(5):755-765). IP₃ sensors that are created to follow intracellular IP₃ changes in single living cells and in cell populations can also be used (Gulyás et al., PLoS ONE, 2015, 10(5):e0125601). Constitutively activated PLCγ2 can also be determined by measuring the increased level of calcium flux stimulated by IP₃ (Novice et al., J. Clin. Immunology, 2020, 40:267-276). Other methods for determining the activity of PLCγ2 are also known in the art and may include detection of PLCγ2 phosphorylation through the use of western blot analysis, immunohistology, and/or enzymatic assays, or detection of the activity of one or more components of the signaling pathway.

In some embodiments, the constitutively activated PLCγ2 is caused by one or more gain-of-function mutations in a gene encoding PLCγ2. Human PLCγ2 (GenBank accession No, P16885, NP_002652.2) is a multidomain protein of 1265 amino acids in length and having the following amino acid sequence:

(SEQ ID NO: 1)
MSTTVNVDSLAEYEKSQIKRALELGTVMTVFSFRKSTPERRTVQVIME
TRQVAWSKTADKIEGFLDIMEIKEIRPGKNSKDFERAKAVRQKEDCCF
TILYGTQFVLSTLSLAADSKEDAVNWLSGLKILHQEAMNASTPTIIES
WLRKQIYSVDQTRRNSISLRELKTILPLINFKVSSAKFLKDKFVEIGA
HKDELSFEQFHLFYKKLMFEQQKSILDEFKKDSSVFILGNTDRPDASA
VYLHDFQRFLIHEQQEHWAQDLNKVRERMTKFIDDTMRETAEPFLFVD
EFLTYLFSRENSIWDEKYDAVDMQDMNNPLSHYWISSSHNTYLTGDQL
RSESSPEAYIRCLRMGCRCIELDCWDGPDGKPVIYHGWTRTTKIKFDD
VVQAIKDHAFVTSSFPVILSIEEHCSVEQQRHMAKAFKEVFGDLLLTK
PTEASADQLPSPSQLREKIIIKHKKLGPRGDVDVNMEDKKDEHKQQGE
LYMWDSIDQKWTRHYCAIADAKLSFSDDIEQTMEEEVPQDIPPTELHF
GEKWFHKKVEKRTSAEKLLQEYCMETGGKDGTFLVRESETFPNDYTLS
FWRSGRVQHCRIRSTMEGGTLKYYLTDNLTFSSIYALIQHYRETHLRC
AEFELRLTDPVPNPNPHESKPWYYDSLSRGEAEDMLMRIPRDGAFLIR
KREGSDSYAITFRARGKVKHCRINRDGRHFVLGTSAYFESLVELVSYY
EKHSLYRKMRLRYPVTPELLERYNMERDINSLYDVSRMYVDPSEINPS
MPQRTVKALYDYKAKRSDELSFCRGALIHNVSKEPGGWWKGDYGTRIQ
QYFPSNYVEDISTADFEELEKQIIEDNPLGSLCRGILDLNTYNVVKAP
QGKNQKSFVFILEPKQQGDPPVEFATDRVEELFEWFQSIREITWKIDT
KENNMKYWEKNQSIAIELSDLVVYCKPTSKTKDNLENPDFREIRSFVE
TKADSIIRQKPVDLLKYNQKGLTRVYPKGQRVDSSNYDPFRLWLCGSQ
MVALNFQTADKYMQMNHALFSLNGRTGYVLQPESMRTEKYDPMPPESQ
RKILMTLTVKVLGARHLPKLGRSIACPFVEVEICGAEYDNNKFKTTVV
NDNGLSPIWAPTQEKVTFEIYDPNLAFLRFVVYEEDMFSDPNFLAHAT
YPIKAVKSGFRSVPLKNGYSEDIELASLLVFCEMRPVLESEEELYSSC
RQLRRRQEELNNQLFLYDTHQNLRNANRDALVKEFSVNENQLQLYQEK
CNKRLREKRVSNSKFYS.

The gene encoding human PLCγ2 is located on Chromosome 16 and contains 33 exons (HGNC:9066). The mRNA (GenBank accession No. NM_002661.5) contains 8666 nucleotides and the coding region is located at nucleotides 182-3979 having the following nucleotide sequence:

(SEQ ID NO: 2)
atgtccaccacggtcaatgtagattcccttgcggaatatgagaagagc
cagatcaagagagccctggagctggggacggtgatgactgtgttcagc
ttccgcaagtccacccccgagcggagaaccgtccaggtgatcatggag
acgcggcaggtggcctggagcaagaccgctgacaagatcgagggcttc
ttggatatcatggaaataaaagaaatccgcccagggaagaactccaaa
gatttcgagcgagcaaaagcagttcgccagaaagaagactgctgcttc
accatcctatatggcactcagttcgtcctcagcacgctcagcttggca
gctgactctaaagaggatgcagttaactggctctctggcttgaaaatc
ttacaccaggaagcgatgaatgcgtccacgcccaccattatcgagagt
tggctgagaaagcagatatattctgtggatcaaaccagaagaaacagc
atcagtctccgagagttgaagaccatcttgcccctgatcaactttaaa
gtgagcagtgccaagttccttaaagataagtttgtggaaataggagca
cacaaagatgagctcagctttgaacagttccatctcttctataaaaaa
cttatgtttgaacagcaaaaatcgattctcgatgaattcaaaaaggat
tcgtccgtgttcatcctggggaacactgacaggccggatgcctctgct
gtttacctgcatgacttccagaggtttctcatacatgaacagcaggag
cattgggctcaggatctgaacaaagtccgtgagcggatgacaaagttc
attgatgacaccatgcgtgaaactgctgagcctttcttgtttgtggat
gagttcctcacgtacctgttttcacgagaaaacagcatctgggatgag
aagtatgacgcggtggacatgcaggacatgaacaaccccctgtctcat
tactggatctcctcgtcacataacacgtaccttacaggtgaccagctg
cggagcgagtcgtccccagaagcttacatccgctgcctgcgcatgggc
tgtcgctgcattgaactggactgctgggacgggcccgatgggaagccg
gtcatctaccatggctggacgcggactaccaagatcaagtttgacgac
gtcgtgcaggccatcaaagaccacgcctttgttacctcgagcttccca
gtgatcctgtccatcgaggagcactgcagcgtggagcaacagcgtcac
atggccaaggccttcaaggaagtatttggcgacctgctgttgacgaag
cccacggaggccagtgctgaccagctgccctcgcccagccagctgcgg
gagaagatcatcatcaagcataagaagctgggcccccgaggcgatgtg
gatgtcaacatggaggacaagaaggacgaacacaagcaacagggggag
ctgtacatgtgggattccattgaccagaaatggactcggcactactgc
gccattgccgatgccaagctgtccttcagtgatgacattgaacagact
atggaggaggaagtgccccaggatataccccctacagaactacatttt
ggggagaaatggttccacaagaaggtggagaagaggacgagtgccgag
aagttgctgcaggaatactgcatggagacggggggcaaggatggcacc
ttcctggttcgggagagcgagaccttccccaatgactacaccctgtcc
ttctggcggtcaggccgggtccagcactgccggatccgctccaccatg
gagggcgggaccctgaaatactacttgactgacaacctcaccttcagc
agcatctatgccctcatccagcactaccgcgagacgcacctgcgctgc
gccgagttcgagctgcggctcacggaccctgtgcccaaccccaacccc
cacgagtccaagccgtggtactatgacagcctgagccgcggagaggca
gaggacatgctgatgaggattccccgggacggggccttcctgatccgg
aagcgagaggggagcgactcctatgccatcaccttcagggctaggggc
aaggtaaagcattgtcgcatcaaccgggacggccggcactttgtgctg
gggacctccgcctattttgagagtctggtggagctcgtcagttactac
gagaagcattcactctaccgaaagatgagactgcgctaccccgtgacc
cccgagctcctggagcgctacaatatggaaagagatataaactccctc
tacgacgtcagcagaatgtatgtggatcccagtgaaatcaatccgtcc
atgcctcagagaaccgtgaaagctctgtatgactacaaagccaagcga
agcgatgagctgagcttctgccgtggtgccctcatccacaatgtctcc
aaggagcccgggggctggtggaaaggagactatggaaccaggatccag
cagtacttcccatccaactacgtcgaggacatctcaactgcagacttc
gaggagctagaaaagcagattattgaagacaatcccttagggtctctt
tgcagaggaatattggacctcaatacctataacgtcgtgaaagcccct
cagggaaaaaaccagaagtcctttgtcttcatcctggagcccaagcag
cagggcgatcctccggtggagtttgccacagacagggtggaggagctc
tttgagtggtttcagagcatccgagagatcacctggaagattgacacc
aaggagaacaacatgaagtactgggagaagaaccagtccatcgccatc
gagctctctgacctggttgtctactgcaaaccaaccagcaaaaccaag
gacaacttagaaaatcctgacttccgagaaatccgctcctttgtggag
acgaaggctgacagcatcatcagacagaagcccgtcgacctcctgaag
tacaatcaaaagggcctgacccgcgtctacccaaagggacaaagagtt
gactcttcaaactacgaccccttccgcctctggctgtgcggttctcag
atggtggcactcaatttccagacggcagataagtacatgcagatgaat
cacgcattgttttctctcaatgggcgcacgggctacgttctgcagcct
gagagcatgaggacagagaaatatgacccgatgccacccgagtcccag
aggaagatcctgatgacgctgacagtcaaggttctcggtgctcgccat
ctccccaaacttggacgaagtattgcctgtccctttgtagaagtggag
atctgtggagccgagtatgacaacaacaagttcaagacgacggttgtg
aatgataatggcctcagccctatctgggctccaacacaggagaaggtg
acatttgaaatttatgacccaaacctggcatttctgcgctttgtggtt
tatgaagaagatatgttcagcgatcccaactttcttgctcatgccact
taccccattaaagcagtcaaatcaggattcaggtccgttcctctgaag
aatgggtacagcgaggacatagagctggcttccctcctggttttctgt
gagatgcggccagtcctggagagcgaagaggaactttactcctcctgt
cgccagctgaggaggcggcaagaagaactgaacaaccagctctttctg
tatgacacacaccagaacttgcgcaatgccaaccgggatgccctggtt
aaagagttcagtgttaatgagaaccagctccagctgtaccaggagaaa
tgcaacaagaggttaagagagaagagagtcagcaacagcaagttttac
tcatag.

The full sequence of this database sequence corresponding to the mRNA encoding PLCγ2 (NM_002661.5), though not shown here, is incorporated by reference herein. The locations of the 33 exons within the mRNA encoding PLCγ2 are as follows:

| Exon | Nucleotides |
|---|---|
| 1 | 1-134 |
| 2 | 135-374 |
| 3 | 375-518 |
| 4 | 519-612 |
| 5 | 613-660 |
| 6 | 661-745 |
| 7 | 745-829 |
| 8 | 830-873 |
| 9 | 874-946 |
| 10 | 947-1048 |
| 11 | 1049-1167 |
| 12 | 1168-1253 |
| 13 | 1254-1374 |
| 14 | 1375-1543 |
| 15 | 1544-1648 |
| 16 | 1649-1738 |
| 17 | 1739-1914 |
| 18 | 1915-2115 |
| 19 | 2116-2235 |
| 20 | 2236-2416 |
| 21 | 2417-2488 |
| 22 | 2489-2598 |
| 23 | 2599-2695 |
| 24 | 2696-2762 |
| 25 | 2763-2920 |
| 26 | 2921-3023 |
| 27 | 2024-3233 |
| 28 | 3234-3379 |
| 29 | 3380-3494 |
| 30 | 3495-3662 |
| 31 | 3663-3751 |
| 32 | 3752-3936 |
| 33 | 3937-8666 |

A number of gain-of-function mutations in PLCγ2 are known in the art and have been associated with diverse pathologies. For instance, gain-of-function somatic mutations of PLCγ2 associated with cancers can arise following BTK inhibition (e.g., using BTK inhibitor ibrutinib) to treat chronic lymphocytic leukemia, leading to constitutive downstream signaling and lymphocyte proliferation. Such gain-of-function somatic mutations of PLCγ2 associated with cancers may include, but are not limited to, D334H (Maddocks et al., JAMA Oncol., 2015, 1(1):80-87), P664S (Ahn et al., Blood, 2017, 129(11):1469-1479), R665W (Woyach et al., N. Engl. J. Med., 2014, 370:2286-2294), S707Y (Woyach et al., N. Engl. J. Med., 2014, 370:2286-2294), S707P (Maddocks et al., JAMA Oncol., 2015, 1(1):80-87), S707F (Maddocks et al., JAMA Oncol., 2015, 1(1):80-87), A708P (Jones et al., Leukemia, 2017, 31:1645-1647), R742P (Maddocks et al., JAMA Oncol., 2015, 1(1):80-87), L845F (Woyach et al., N. Engl. J. Med., 2014, 370:2286-2294), L845V (Jones et al., Leukemia, 2017, 31:1645-1647), D993Y (Woyach et al., N. Engl. J. Med., 2014, 370:2286-2294), D993H (Jones et al., Leukemia, 2017, 31:1645-1647), D1140G (Maddocks et al., JAMA Oncol., 2015, 1(1):80-87), D1140Y (Jones et al., Leukemia, 2017, 31:1645-1647), D1140N (Jones et al., Leukemia, 2017, 31:1645-1647), D1140E (Jones et al., Leukemia, 2017, 31:1645-1647), D1140V (Jones et al., Leukemia, 2017, 31:1645-1647), M1141R (Burger et al., Nature Communications, 2016, 7:11589), F1142L (Jones et al., Leukemia, 2017, 31:1645-1647), M1141K (Burger et al., Nature Communications, 2016, 7:11589), D1144N (Jones et al., Leukemia, 2017, 31:1645-1647), D1144G (Jones et al., Leukemia, 2017, 31:1645-1647) mutations in human PLCγ2 (SEQ ID NO: 1). In addition, gain-of-function mutations associated with cancers caused by partial deletions of the PLCγ2 gene have also been reported, including a 6-nucleotide deletion in exon 20 of the PLCγ2 gene (c.2120-2125del), which leads to deletion of S707 and A708 of SEQ ID NO: 1 (Ahn et al., Blood, 2017, 129(11):1469-1479).

Likewise, inherited gain-of-function germline heterozygous mutations of PLCγ2 that lead to diverse immune dysregulations are also known in the art, and include, but are not limited to, P139S (Kutukculer et al., Clin. Case Rep., 2021, 9:2023-2031), T168A (Kutukculer et al., Clin. Case Rep., 2021, 9:2023-2031), I169V (Wu et al., Front. Immunol., 2021, 12:667430), Y482H (Kutukculer et al., Clin. Case Rep., 2021, 9:2023-2031), N571S (Kutukculer et al., Clin. Case Rep., 2021, 9:2023-2031), S707Y (Zhou et al., Am. J. Hum. Genet., 2012, 91:713-720), S707P (Park et al., European Journal of Medical Genetics, 2022, 65(1):104387), A708P (Martin-Nalda et al, Annual Meeting of European Society for Immunodeficiencies, 2017), S718R (Kutukculer et al., Clin. Case Rep., 2021, 9:2023-2031), L848P (Neves et al., Front. Immunol., 2018, 9:2863; Park et al., European Journal of Medical Genetics, 2022, 65(1):104387), M1141L (Jackson et al., J. Biol. Chem., 2021, 297(2):100905), M1141K (Novice et al., Journal of Clinical Immunology, 2020, 40:267-276) mutations in human PLCγ2 (SEQ ID NO: 1). In addition, gain-of-function mutations associated with immune dysregulations caused by partial deletions of the PLCγ2 gene were also reported, including exon 19 deletion (Ombrello et al., N. Engl. J. Med., 2012, 366:330-338), exon 20-22 deletion (Ombrello et al., N. Engl. J. Med., 2012, 366:330-338), and deletion of L845 through L848 of SEQ ID NO: 1 (Martin-Nalda et al, Annual Meeting of European Society for Immunodeficiencies, 2017).

Accordingly, in some embodiments, the one or more gain-of-function mutations in the gene encoding PLCγ2 leading to the constitutively activated PLCγ2 comprise a deletion of one or more nucleotides in the PLCγ2 gene and/or one or more mutations at P139, T168, I169, D334, Y482, N571, P664, R665, S707, A708, S718, R742, L845, L848, D993, D1140, M1141, F1142, or D1144 of SEQ ID NO: 1. In some embodiments, the one or more gain-of-function mutations in the gene encoding PLCγ2 leading to the constitutively activated PLCγ2 comprise one or more of the following mutations: P139S, T168A, I169V, D334H, Y482H, N571S, P664S, R665W, S707Y, S707P, S707F, A708P, S718R, R742P, L845F, L845V, L848P, D993Y, D993H, D1140G, D1140Y, D1140N, D1140E, D1140V, M1141L, M1141R, M1141K, F1142L, D1144N, or D1144G of SEQ ID NO: 1, or a deletion of at least amino acids S707-A708 of PLCγ2 (SEQ ID NO: 1), or a deletion of at least amino acids L845-L848 of PLCγ2 (SEQ ID NO: 1), or a deletion of one or more nucleotides in exons 19-22 of the gene encoding PLCγ2. In some embodiments, the deletion is a partial deletion of exon 19-22. In some embodiments, the deletion is a deletion of the entire exon 19, or exons 20-22. In some embodiments, the deletion is a full deletion of the entire exon 19 of the PLCγ2 gene. In some embodiments, the deletion is a full deletion of the entire exons 20-22 of the PLCγ2 gene.

In some embodiments, the one or more gain-of-function mutations in the gene encoding PLCγ2 leading to the constitutively activated PLCγ2 comprise one or more of the following mutations: D334H, P664S, R665W, S707Y, S707P, S707F, A708P, R742P, L845F, L845V, D993Y, D993H, D1140G, D1140Y, D1140N, D1140E, D1140V, M1141R, M1141K, F1142L, D1144N, or D1144G of SEQ ID NO: 1, or a deletion of at least S707-A708 of PLCγ2 (SEQ ID NO: 1), or a deletion of one or more nucleotides in exon 20 of the gene encoding PLCγ2, and the disease or disorder associated with the constitutively activated PLCγ2 is a cancer. In other embodiments, the one or more gain-of-function mutations in the gene encoding PLCγ2 leading to the constitutively activated PLCγ2 comprise one or more of the following mutations: P139S, T168A, I169V, Y482H, N571S, S707Y, S707P, A708P, S718R, L848P, M1141L, or M1141K of SEQ ID NO: 1, or a deletion of at least amino acids L845-L848 of PLCγ2 (SEQ ID NO: 1), or a deletion of one or more nucleotides of exons 19-22 of the gene encoding PLCγ2, such as a full deletion of the entire exon 19 or a full deletion of the entire exons 20-22 of the PLCγ2 gene, and the disease or disorder associated with the constitutively activated PLCγ2 is an immune system disorder.

Human PLCγ2 is a multidomain protein characterized by a N-terminal pleckstrin homology (PH) domain (amino acid residues 15-134 of SEQ ID NO: 1), an EF-hand domain (amino acid residues 138-294 of SEQ ID NO: 1), a catalytic domain, and a calcium binding (C2) domain (amino acid residues 1061-1187 of SEQ ID NO: 1). In addition, and specific to the PLCγ family, PLCγ2 has a specific array of domains (7SA) inserted through a loop in the catalytic domain (amino acid residues 320-454 and 928-1027 of SEQ ID NO: 1) comprising a “split PH domain,” two SH2 domains (amino acid residues 531-617 and 646-735 of SEQ ID NO: 1) and one SH3 domain (amino acid residues 772-827 of SEQ ID NO: 1). This multidomain insert (amino acid residues 477-907 of SEQ ID NO: 1) in the catalytic domain constitutes the regulatory domain of PLCγ2. See e.g., Magno et al., Molecular Neurodegeneration, 2121, 16:22. It has been reported that PLAID-causing genomic deletions (Δ19 and Δ20-22) and APLAID-associated somatic mutations are located within the regulatory domain (e.g., S707Y, L848P, A708P of SEQ ID NO: 1) or in the calcium binding domain (e.g., M1141L of SEQ ID NO: 1) of PLCγ2, and acquired mutations as a result of BTK inhibition (e.g., S707Y, L845F of SEQ ID NO: 1) are found in the regulatory domain of PLCγ2. See e.g., Jackson et al., J. Biol. Chem., 2021, 297(2):100905. Thus, in some embodiments, the one or more gain-of-function mutations in the gene encoding PLCγ2 leading to the constitutively activated PLCγ2 are located within the regulatory domain and/or the calcium binding domain of PLCγ2. The “regulatory domain” and the “calcium binding domain,” as used herein, refers to the region located within, or in the close vicinity (e.g., plus/minus 6 amino acid residues) of amino acid residues 477-907 and amino acid residues 1061-1187 of the human PLCγ2 (SEQ ID NO: 1), respectively. In some embodiments, the one or more gain-of-function mutations located within the regulatory domain and/or the calcium binding domain of PLCγ2 comprise one or more mutations at Y482, N571, P664, R665, S707, A708, S718, R742, L845, L848, D1140, M1141, F1142, or D1144 of SEQ ID NO: 1. In some embodiments, the one or more gain-of-function mutations located within the regulatory domain and/or the calcium binding domain of PLCγ2 comprise one or more of the following mutations: Y482H, N571S, P664S, R665W, S707Y, S707P, S707F, A708P, S718R, R742P, L845F, L845V, L848P, D993Y, D993H, D1140G, D1140Y, D1140N, D1140E, D1140V, M1141L, M1141R, M1141K, F1142L, D1144N, or D1144G of SEQ ID NO: 1.

A gain-of-function mutation in PLCγ2 typically leads to a constitutive phospholipase activity, as measured by standard enzymatic assays as described elsewhere herein, such as that described in Everett et al. (J. Biol. Chem., 2009, 284(34):23083-23093), Woyach et al. (N. Engl. J. Med., 2014, 370:2286-2294), or Novice et al. (J. Clin. Immunology, 2020, 40:267-276), which in turn may result in a gain-of-function phenotype due to the constitutively activated PLCγ2. In some situations, however, such as in certain non-malignant immune cells, a constitutively activated PLCγ2 may lead to decreased PLCγ2-dependent signaling and function, i.e., loss of PLCγ2 downstream function, which may be a direct result of chronic signaling, similar to how chronic B-cell-receptor stimulation results in calcium currents of diminished amplitude, alteration of signaling cascades, and ultimately, proliferative anergy (Ombrello et al., N. Engl. J. Med., 2012, 366:330-338). There are several possible explanations to the mechanisms underlying the reduction in PLCγ2-mediated signal transduction. For instance, increased phospholipase activity may lead to depletion of one or more substrates, such as the substrate PIP2 proximate to PLCγ2, resulting in impaired IP3 production and IP3-mediated calcium release. Alternatively, excessively high concentrations of the products of PLCγ2 may induce feedback-mediated down-regulation of the distal signaling pathways, resulting in the induction of anergy. Nevertheless, the specific mechanisms that lead to the reduction in PLCγ2-mediated signal transduction remain to be elucidated. See, Ombrello et al., N. Engl. J. Med., 2012, 366:330-338.

In some embodiments, therefore, the one or more gain-of-function mutations in PLCγ2 may lead to a gain-of-function phenotype in the subject to be treated by the method disclosed herein. In other embodiments, the one or more gain-of-function mutations in PLCγ2 may lead to a loss-of-function phenotype in the subject to be treated by the method disclosed herein.

### BTK Reducing Molecules

Any molecule that can reduce or eliminate the amount of BTK in a cell can be used as the BTK reducing molecule in any of the methods disclosed herein. Reducing or eliminating the amount of BTK can be achieved in several ways. For instance, the amount of BTK can be reduced or eliminated using a small molecule that is capable of inducing proteolytic degradation of BTK, such as a proteolysis-targeting chimera (see e.g., Sakamoto et al., PNAS, 2001, 98:8554-8559; Sakamoto et al., Methods Enzymol., 2005, 399:833-847). Alternatively, the amount of BTK can be reduced or eliminated using a nucleic acid molecule that is capable of inducing sequence-specific suppression of BTK gene expression, such as an antisense oligonucleotide, a microRNA, a ribozyme, an antagomir, an aptamer, or a RNAi molecule.

a. BTK Degrader Molecule

In one aspect, the BTK reducing molecule of the disclosure is a BTK degrader molecule, such as a small molecule, that reduces or eliminates the amount of BTK by inducing proteolytic degradation of BTK. Proteolysis-targeting chimera is a novel strategy for selective knockdown of target proteins by small molecules (Sakamoto et al., PNAS, 2001, 98:8554-8559; Sakamoto et al., Methods Enzymol., 2005, 399:833-847). Proteolysis-targeting chimera utilizes the ubiquitin-protease system to target a specific protein and induce its degradation in the cell (Zhou et al., Mol. Cell, 2000, 6(3):751-756; Neklesa et al., Pharmacol. Ther., 2017, 174:138-144; Lu et al., Eur. J. Med. Chem., 2018, 146:251-259). Accordingly, in some embodiments, the BTK degrader molecule of the disclosure is a proteolysis-targeting chimera.

In some embodiments, the BTK degrader molecule useful for the methods of the disclosure is a compound of Formula I as described in PCT Application No. PCT/US22/14830, which is hereby incorporated by reference in its entirety:

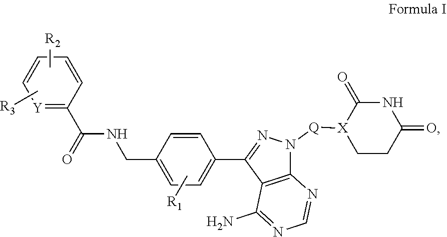

or a pharmaceutically acceptable salt thereof,
wherein X is CH or N;
wherein Y is CH or N;
wherein R₁ is chosen from H, C₁-C₃ alkyl, C₁-C₃ haloalkyl, dialkylamino group, amino group, —CN, hydroxyl, C₁-C₄ alkoxy, and halogen;
wherein each R₂ and R³ is independently chosen from H, halogen, —CN, hydroxyl, dialkylamino group, C₁-C₅ alkyl, deuterated C₁-C₅ alkyl, C₁-C₅ alkoxy, deuterated C₁-C₅ alkoxy, and C₁-C₅ haloalkyl;
wherein Q is L-W₁ or L-W₂;
wherein L is a linker of 2 to 20 carbon atoms in length, wherein one or more carbon atoms are optionally and independently replaced by a group chosen from C(═O), O, N(R₆), S, S(O), SO₂, C(O)NH, C(O)NCH₃, C(O)NCH₂CH₃, C₂-alkenyl, C₂-alkynyl, cycloalkyl, heterocycloalkyl, heterocycle, aryl, or heteroaryl, wherein each are independently substituted with 0, 1, 2 or 3 R₇;
wherein W₁ is chosen from

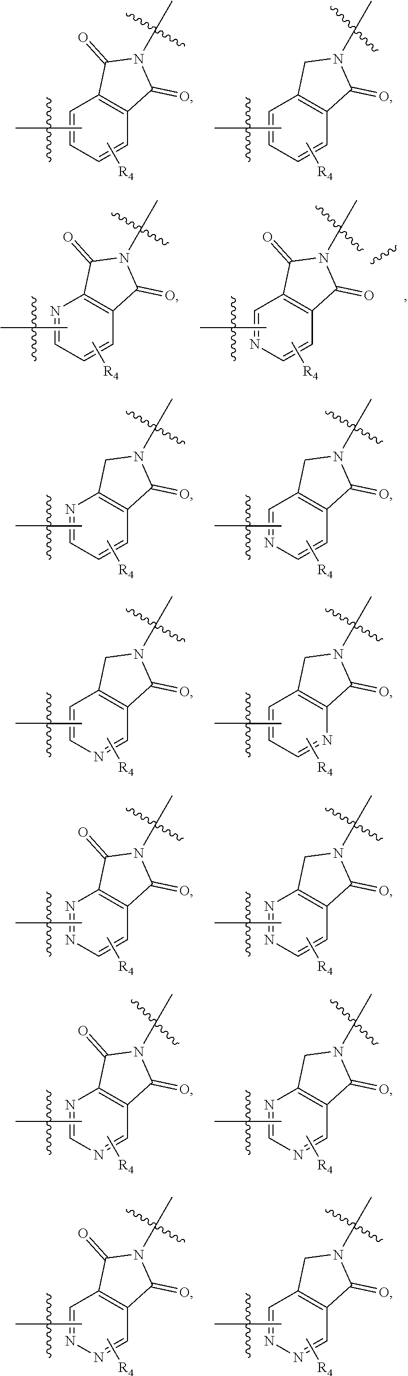

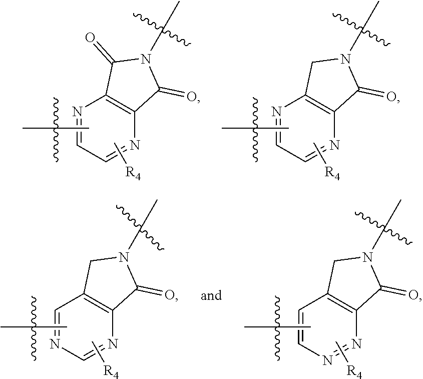

wherein R₄ is chosen from H, halogen, —CN, C₁-C₅ alkyl, C₁-C₅ alkoxy, and C₁-C₅ haloalkyl; and
wherein W₂ is

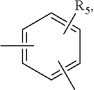

wherein R₅ is chosen from H, halogen, —CN, C₁-C₅ alkyl, deuterated C₁-C₅ alkyl, C₁-C₅ alkoxy, deuterated C₁-C₅ alkoxy, and C₁-C₅ haloalkyl; wherein each R₆ is independently chosen from H, C₁-C₃ alkyl, —C(═O)—(C₁-C₃ alkyl), —C(═O)—O—(C₁-C₃ alkyl), and —C(═O)—NH—(C₁-C₃ alkyl), each of which is substituted with 0, 1, 2, or 3 R₇; and
wherein each R₇ is independently chosen from halogen, hydroxyl, amino group, C₁-C₃ alkyl, C₁-C₃ alkoxy, C₁-C₃ haloalkyl, —N(R₆)2, and —CN.

In some embodiments, L in Formula I is 2 to 12 carbon atoms in length, wherein one or more carbon atoms are optionally and independently replaced by a group selected from C(═O), O, S, S(O), SO₂, C(O)NH, C(O)NCH₃, C(O)NCH₂CH₃, NH, NCH₃, NCH₂CH₃, C₂-alkynyl,

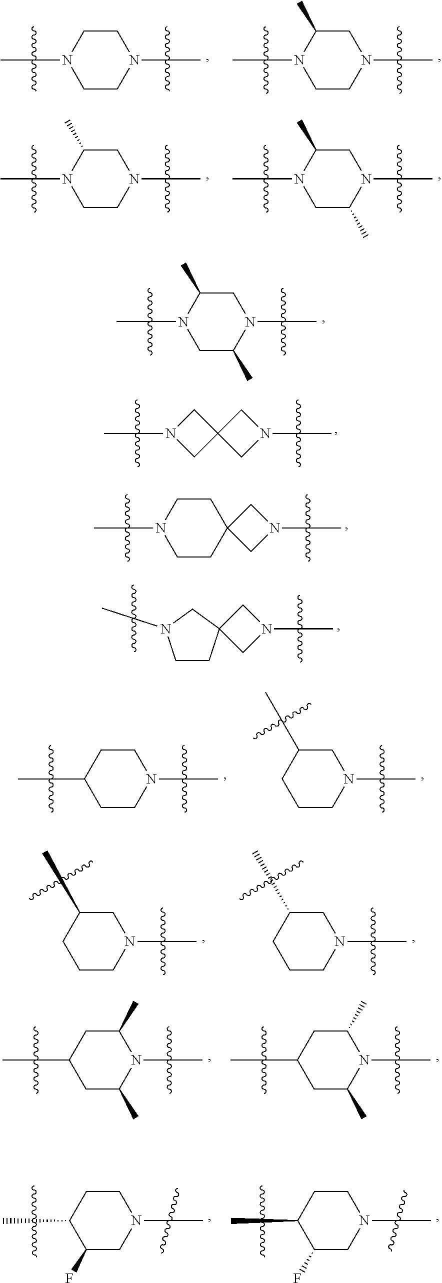

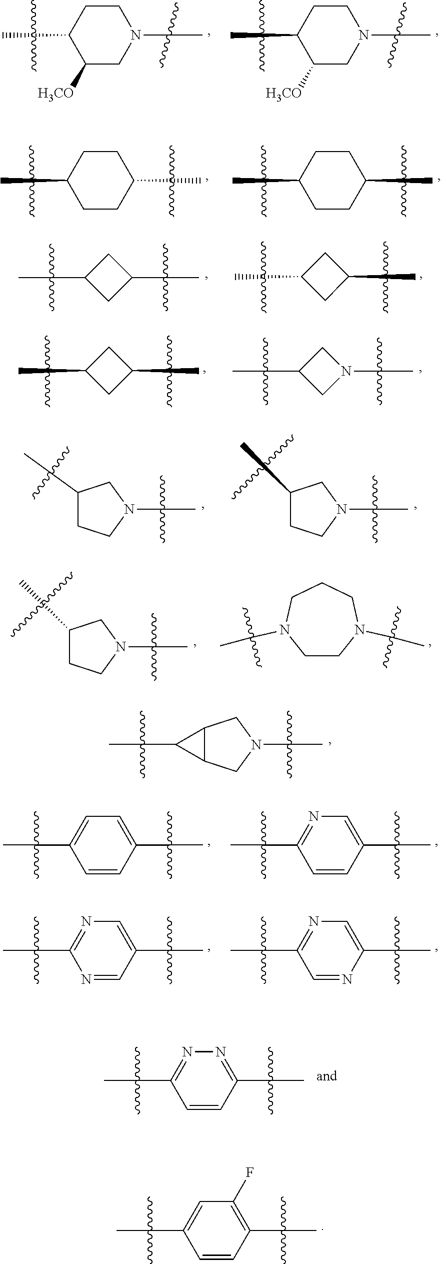

In some embodiments, the BTK degrader molecule useful for the methods of the disclosure is a compound of:

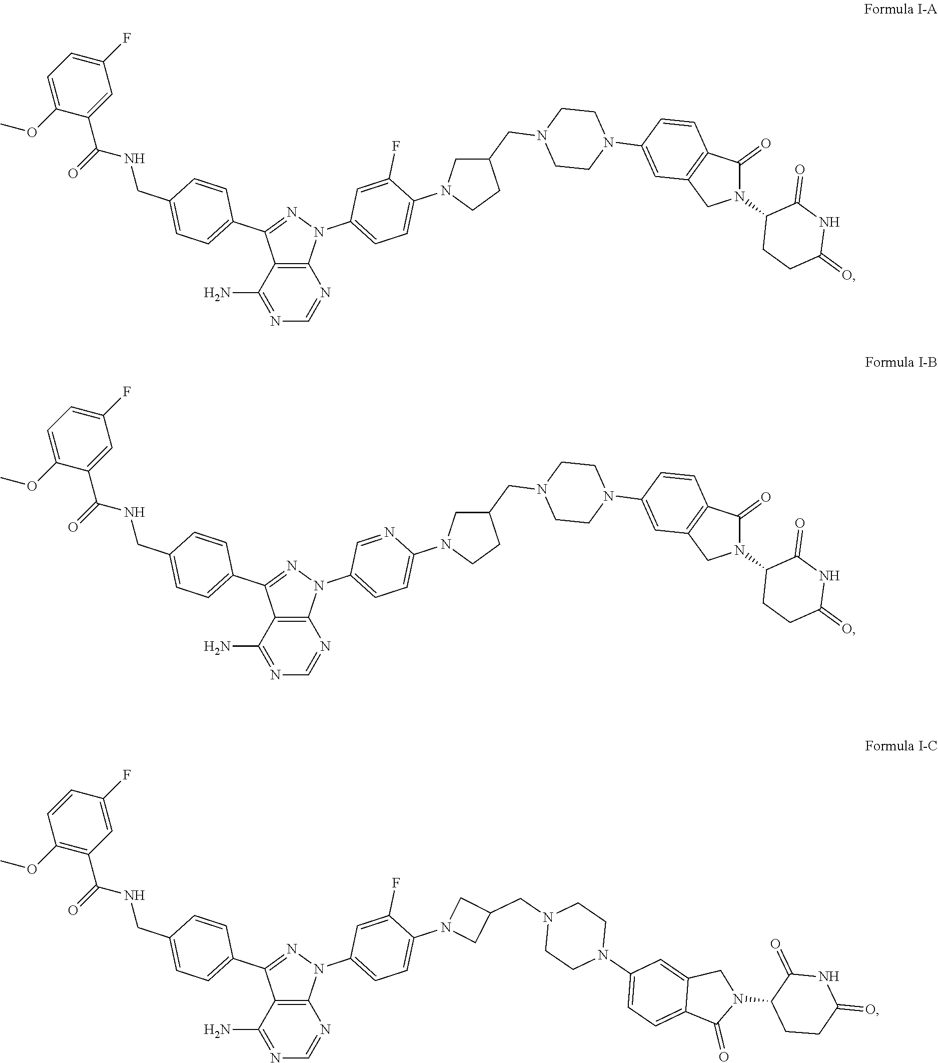 [SMILES: COc1ccc(F)cc1C(=O)NCc1ccc(-c2nn(-c3ccc(N4CCC(CN5CCN(c6ccc7c(c6)CN([C@H]6CCC(=O)NC6=O)C7=O)CC5)C4)c(F)c3)c3ncnc(N)c23)cc1 ; COc1ccc(F)cc1C(=O)NCc1ccc(-c2nn(-c3ccc(N4CCC(CN5CCN(c6ccc7c(c6)CN([C@H]6CCC(=O)NC6=O)C7=O)CC5)C4)nc3)c3ncnc(N)c23)cc1 ; COc1ccc(F)cc1C(=O)NCc1ccc(-c2nn(-c3ccc(N4CC(CN5CCN(c6ccc7c(c6)CN([C@H]6CCC(=O)NC6=O)C7=O)CC5)C4)c(F)c3)c3ncnc(N)c23)cc1]

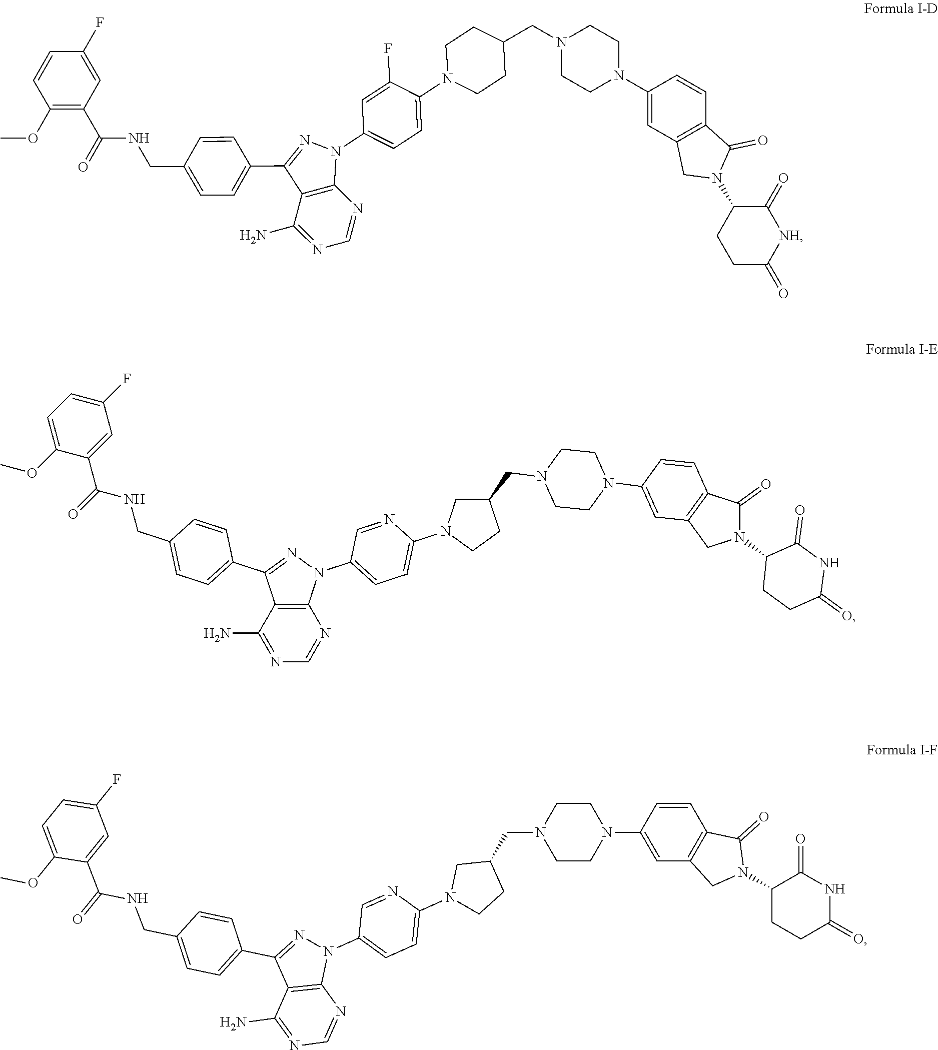 [SMILES: COc1ccc(F)cc1C(=O)NCc1ccc(-c2nn(-c3ccc(N4CCC(CN5CCN(c6ccc7c(c6)CN([C@H]6CCC(=O)NC6=O)C7=O)CC5)CC4)c(F)c3)c3ncnc(N)c23)cc1 ; COc1ccc(F)cc1C(=O)NCc1ccc(-c2nn(-c3ccc(N4CC[C@H](CN5CCN(c6ccc7c(c6)CN([C@H]6CCC(=O)NC6=O)C7=O)CC5)C4)nc3)c3ncnc(N)c23)cc1 ; COc1ccc(F)cc1C(=O)NCc1ccc(-c2nn(-c3ccc(N4CC[C@@H](CN5CCN(c6ccc7c(c6)CN([C@H]6CCC(=O)NC6=O)C7=O)CC5)C4)nc3)c3ncnc(N)c23)cc1]

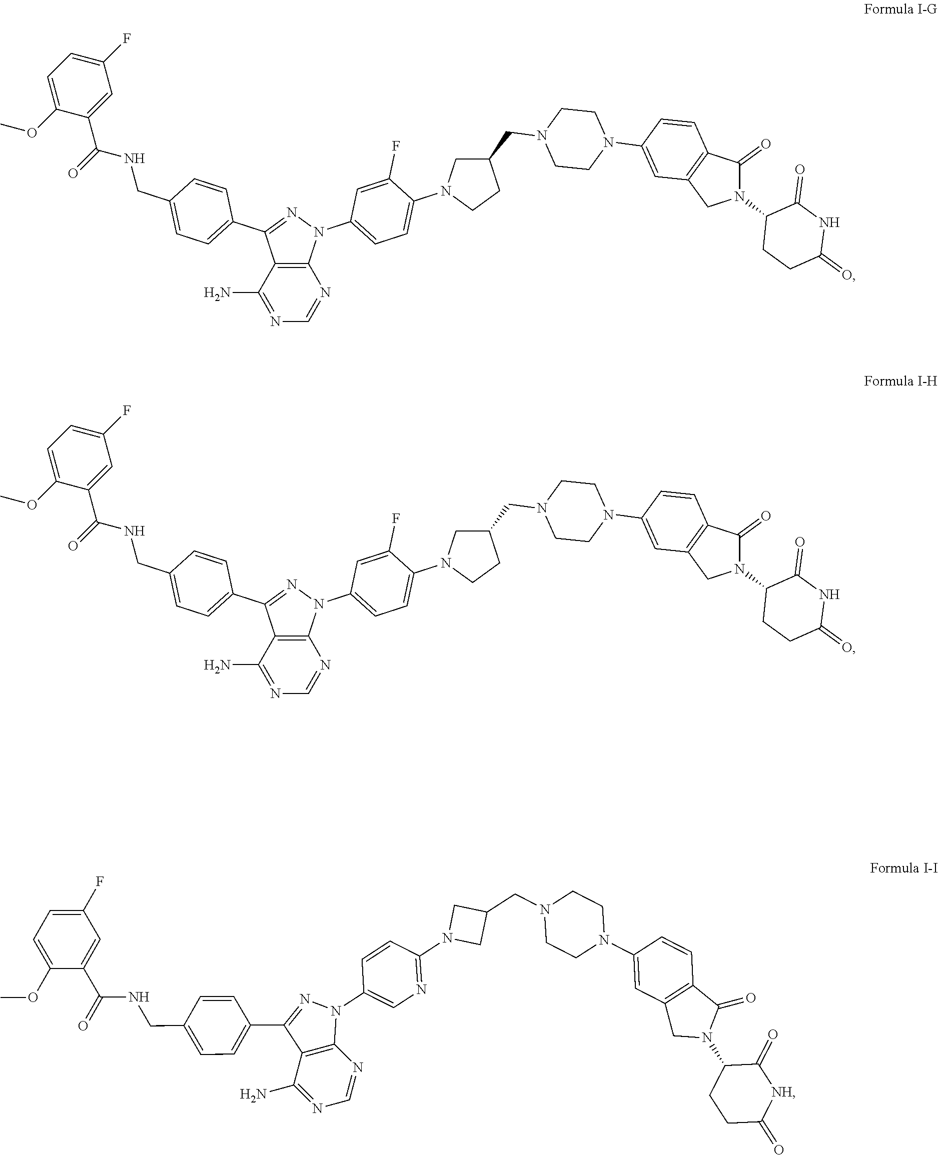 [SMILES: COc1ccc(F)cc1C(=O)NCc1ccc(-c2nn(-c3ccc(N4CC[C@H](CN5CCN(c6ccc7c(c6)CN([C@H]6CCC(=O)NC6=O)C7=O)CC5)C4)c(F)c3)c3ncnc(N)c23)cc1 ; COc1ccc(F)cc1C(=O)NCc1ccc(-c2nn(-c3ccc(N4CC[C@@H](CN5CCN(c6ccc7c(c6)CN([C@H]6CCC(=O)NC6=O)C7=O)CC5)C4)c(F)c3)c3ncnc(N)c23)cc1 ; COc1ccc(F)cc1C(=O)NCc1ccc(-c2nn(-c3ccc(N4CC(CN5CCN(c6ccc7c(c6)CN([C@H]6CCC(=O)NC6=O)C7=O)CC5)C4)nc3)c3ncnc(N)c23)cc1]

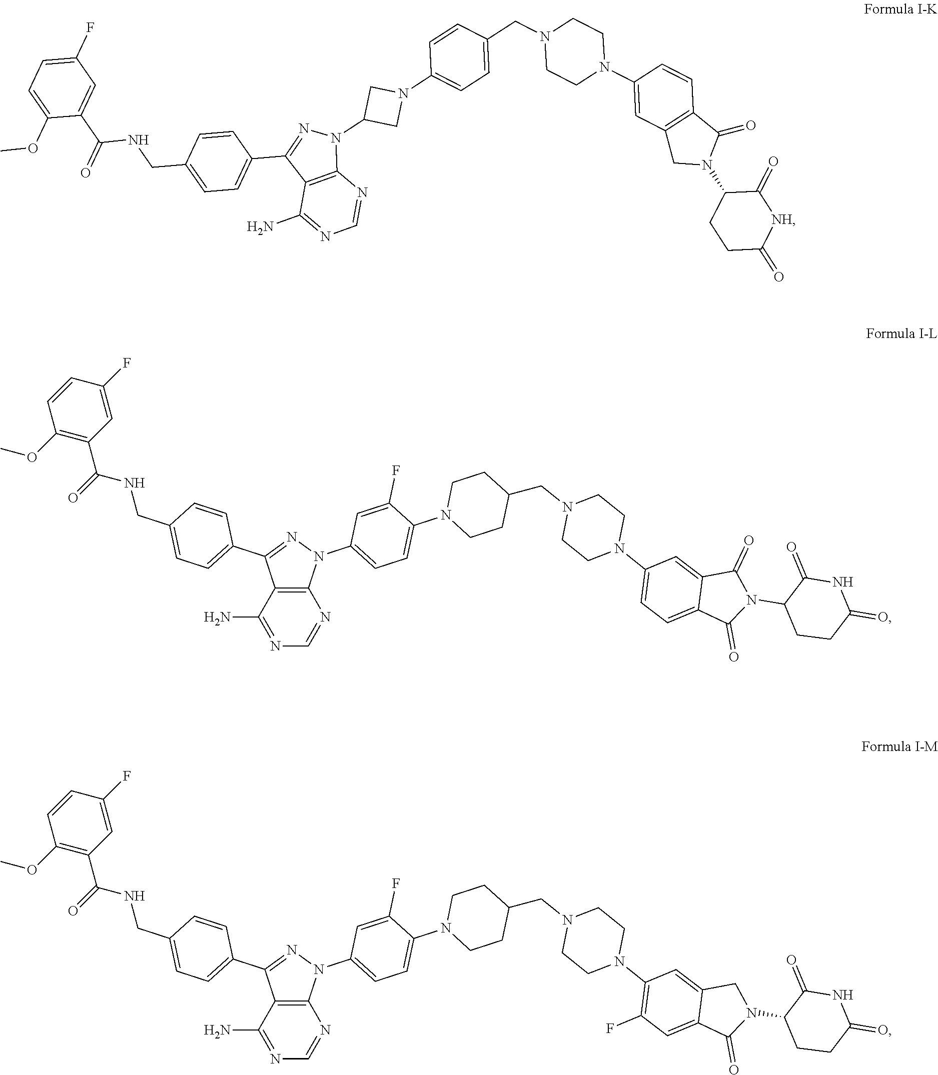 [SMILES: COc1ccc(F)cc1C(=O)NCc1ccc(-c2nn(C3CN(c4ccc(CN5CCN(c6ccc7c(c6)CN([C@H]6CCC(=O)NC6=O)C7=O)CC5)cc4)C3)c3ncnc(N)c23)cc1 ; COc1ccc(F)cc1C(=O)NCc1ccc(-c2nn(-c3ccc(N4CCC(CN5CCN(c6ccc7c(c6)C(=O)N(C6CCC(=O)NC6=O)C7=O)CC5)CC4)c(F)c3)c3ncnc(N)c23)cc1 ; COc1ccc(F)cc1C(=O)NCc1ccc(-c2nn(-c3ccc(N4CCC(CN5CCN(c6cc7c(cc6F)C(=O)N([C@H]6CCC(=O)NC6=O)C7)CC5)CC4)c(F)c3)c3ncnc(N)c23)cc1]

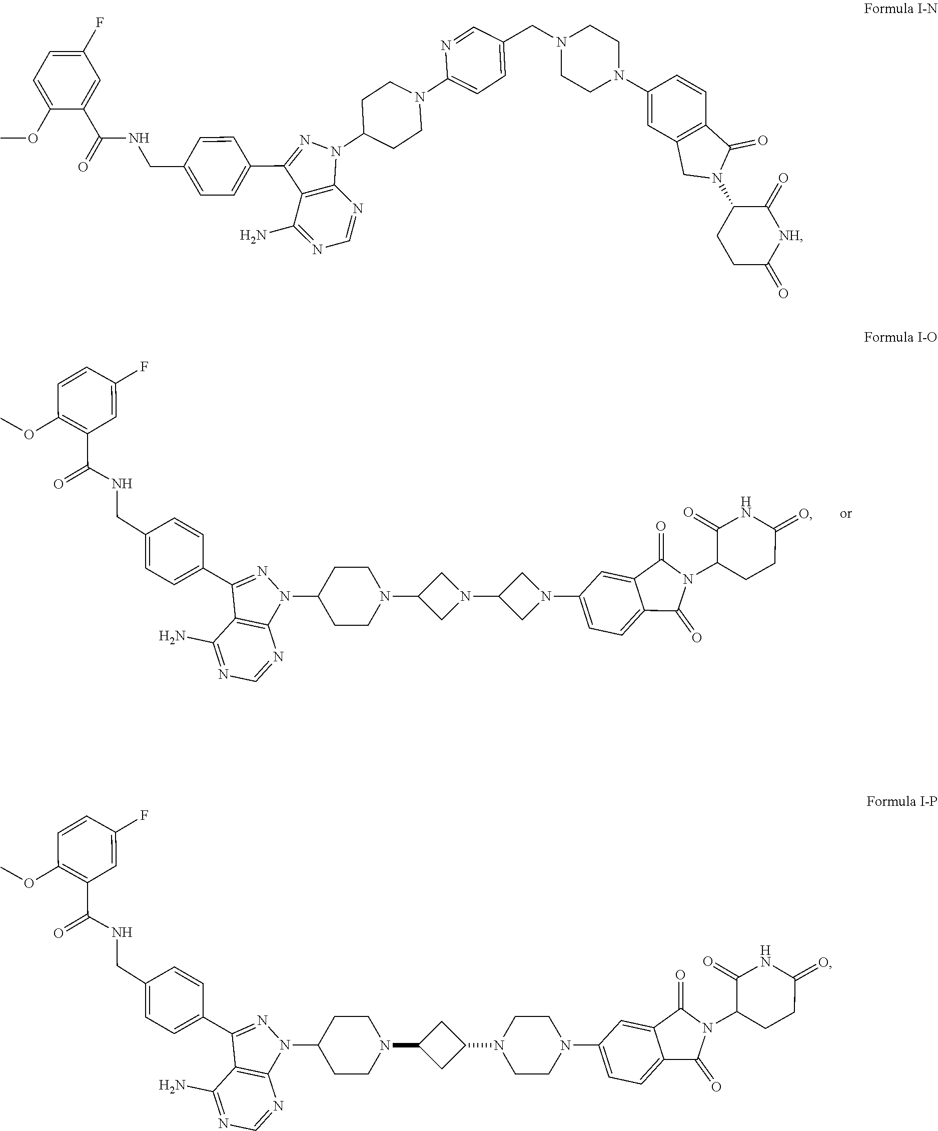

or a pharmaceutically acceptable salt thereof. Methods of preparing compounds of Formula I, such as compounds of Formula I-A to I-P, are described in PCT Application No. PCT/US22/14830, which is hereby incorporated by reference herein in its entirety.

In some embodiments, the BTK degrader molecule useful for the methods of the disclosure is a compound of Formula II, as described in WO 2021/113557, incorporated by reference herein:

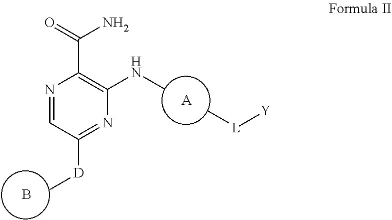

or a pharmaceutically acceptable salt thereof,
wherein W is CH or N;
D is a bond or —NH—;
Ring A is phenyl, substituted phenyl group;
Ring B is a 4-6 membered heterocycloalkyl where the heterocyclic alkyl can be substituted with halogen, CN, and heterocycles where the heterocycles can contain N and or O;
L is C₀₋₁₂ linker where each carbon cab be substituted with —O—, —N(R)—C(O)—, —N(R)—, —C(O)—, —S—, —SO—, SO₂—, —C(O)—N(R)—, 4-6 membered monocyclic cycloalkyl, or 4-6 membered monocyclic heterocycle, where R can be lower alkyl, and lower alkyl substituted with halogen, alkoxy, CN, and hydroxyl groups;
Y is

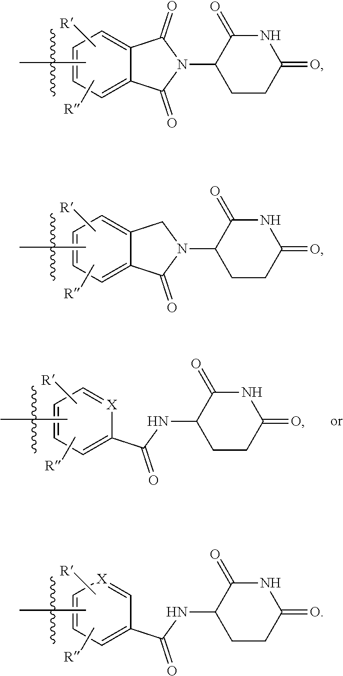

In some embodiments, the BTK degrader molecule useful for the methods of the disclosure is a compound having the following formula as described in WO 2021/113557, incorporated by reference herein:

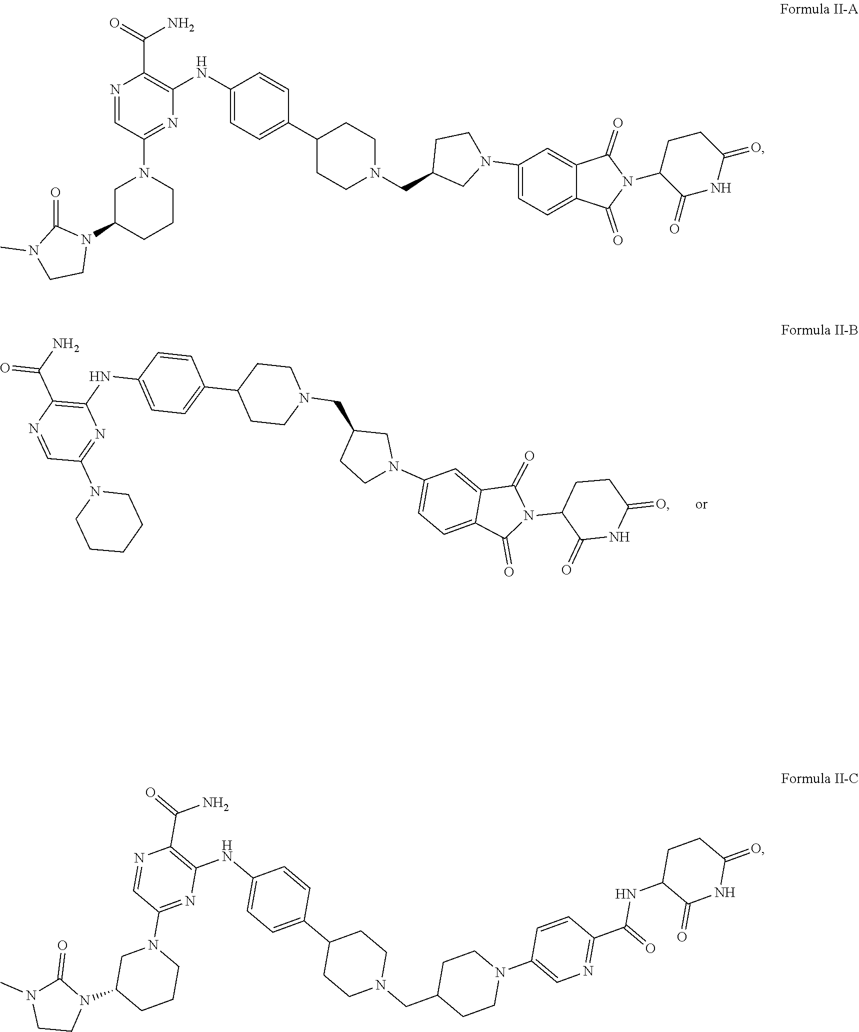 [SMILES: CN1CCN([C@@H]2CCCN(c3cnc(C(N)=O)c(Nc4ccc(C5CCN(C[C@H]6CCN(c7ccc8c(c7)C(=O)N(C7CCC(=O)NC7=O)C8=O)C6)CC5)cc4)n3)C2)C1=O ; NC(=O)c1ncc(N2CCCCC2)nc1Nc1ccc(C2CCN(C[C@@H]3CCN(c4ccc5c(c4)C(=O)N(C4CCC(=O)NC4=O)C5=O)C3)CC2)cc1 ; CN1CCN([C@H]2CCCN(c3cnc(C(N)=O)c(Nc4ccc(C5CCN(CC6CCN(c7ccc(C(=O)NC8CCC(=O)NC8=O)nc7)CC6)CC5)cc4)n3)C2)C1=O]

or a pharmaceutically acceptable salt thereof.

In some embodiments, the BTK degrader molecule useful for the methods of the disclosure is a compound of Formula III, as described in WO 2020/239103, incorporated by reference herein:

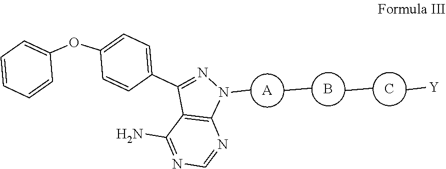

or a pharmaceutically acceptable salt thereof,
wherein A, B and C are independent cyclic or heterocyclic structures with the ring size as 4 to 7 atoms, the heterocyclic structures can be the following:

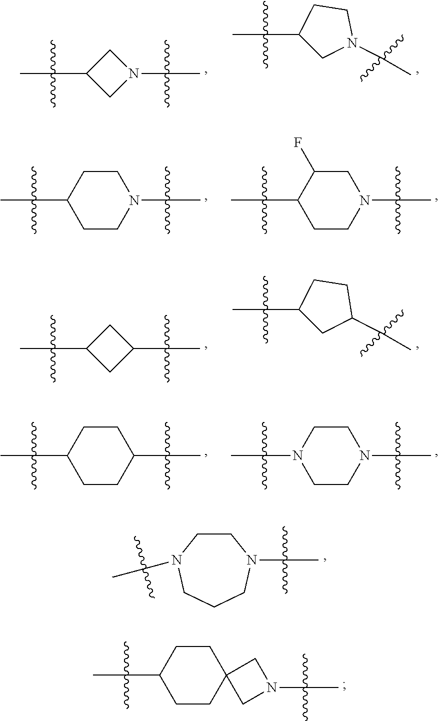

wherein Y is

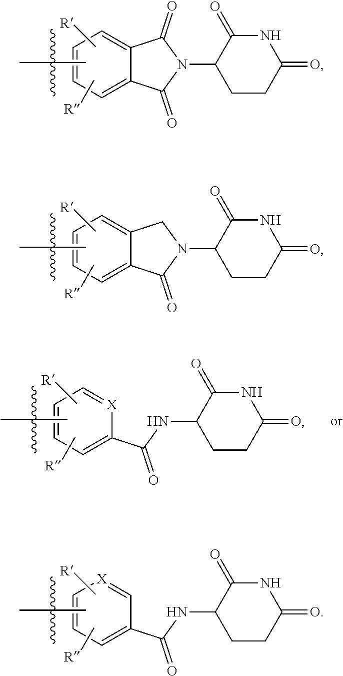

In some embodiments, the BTK degrader molecule useful for the methods of the disclosure is a compound having the following formula as described in WO 2020/239103, incorporated by reference herein:

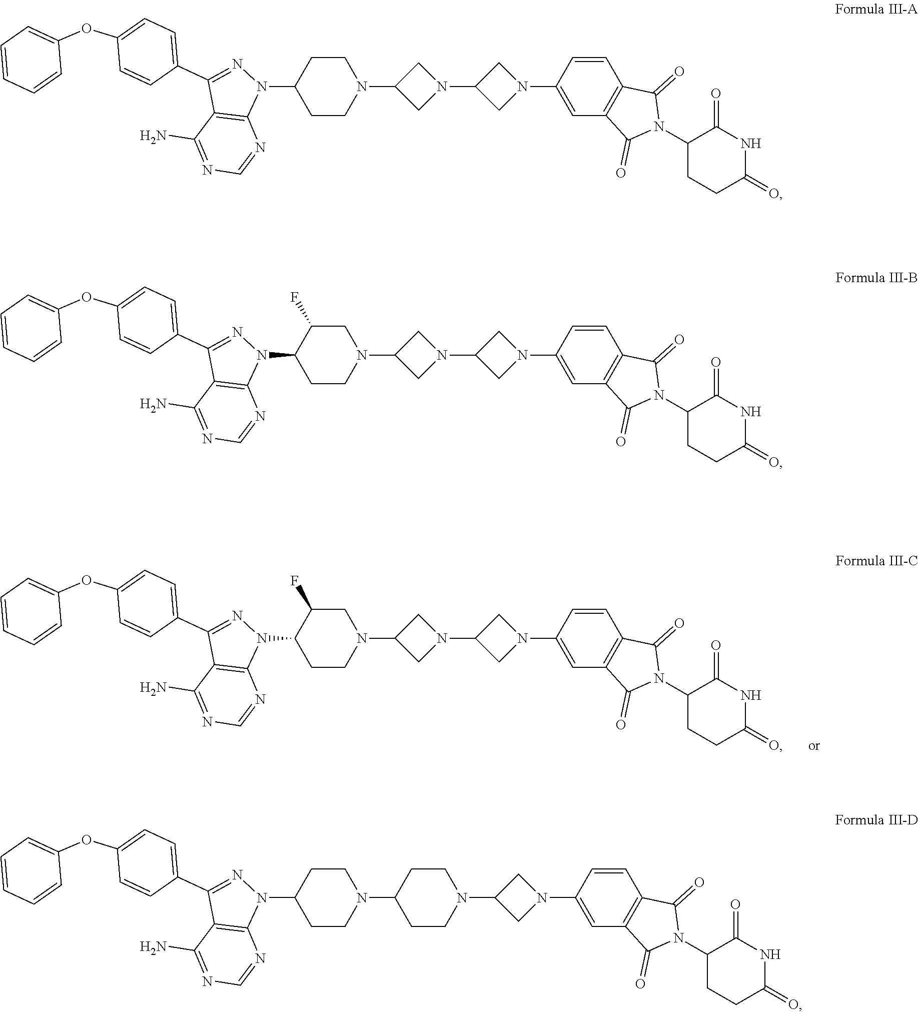 [SMILES: Nc1ncnc2c1c(-c1ccc(Oc3ccccc3)cc1)nn2C1CCN(C2CN(C3CN(c4ccc5c(c4)C(=O)N(C4CCC(=O)NC4=O)C5=O)C3)C2)CC1 ; Nc1ncnc2c1c(-c1ccc(Oc3ccccc3)cc1)nn2[C@@H]1CCN(C2CN(C3CN(c4ccc5c(c4)C(=O)N(C4CCC(=O)NC4=O)C5=O)C3)C2)C[C@H]1F ; Nc1ncnc2c1c(-c1ccc(Oc3ccccc3)cc1)nn2[C@H]1CCN(C2CN(C3CN(c4ccc5c(c4)C(=O)N(C4CCC(=O)NC4=O)C5=O)C3)C2)C[C@@H]1F ; Nc1ncnc2c1c(-c1ccc(Oc3ccccc3)cc1)nn2C1CCN(C2CCN(C3CN(c4ccc5c(c4)C(=O)N(C4CCC(=O)NC4=O)C5=O)C3)CC2)CC1]

or a pharmaceutically acceptable salt thereof.

In some embodiments, the BTK degrader molecule useful for the methods of the disclosure is a compound of Formula IV, as described in WO 2021/219071, incorporated by reference herein:

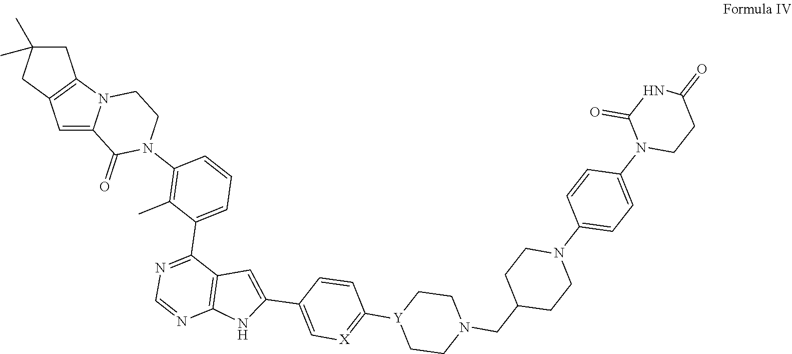

Wherein X and Y can independently be CH or N,
or a pharmaceutically acceptable salt thereof.

The BTK degrader molecule of Formula 1 can be synthesized according to Scheme 1, 2, 3 and 4 below.

Scheme 1: Synthesis of BTK degrader molecules with Formula I-A to Formula I-J, Formula I-L and Formula I-M.

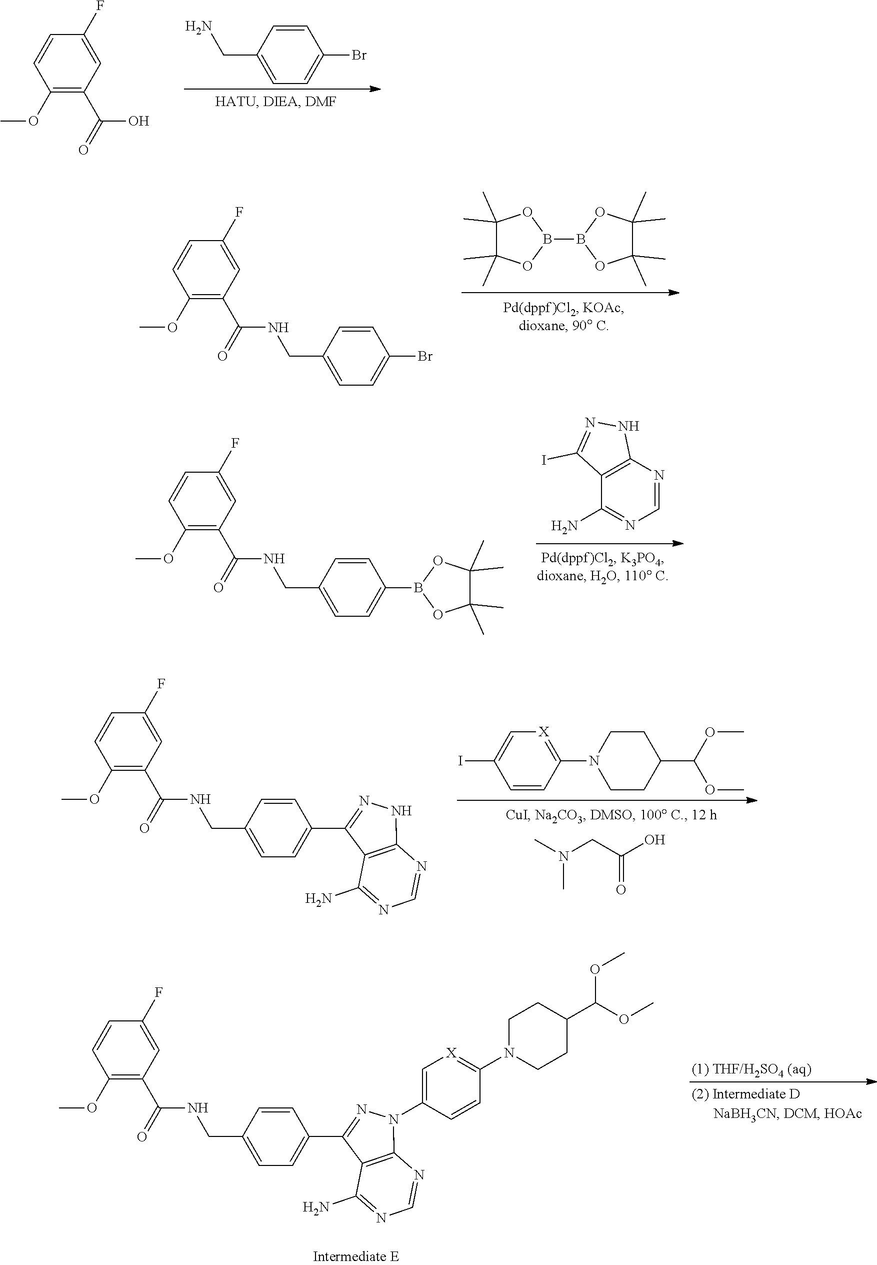

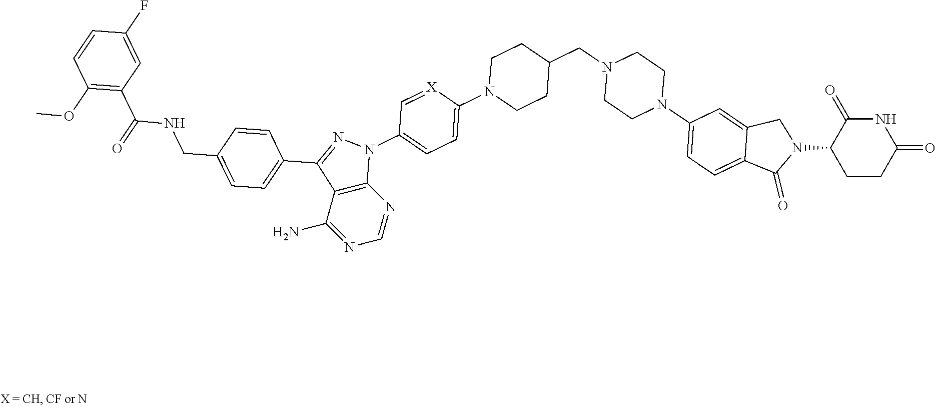

Scheme 2: Synthesis of BTK degrader molecules with Formula I-K and Formula I-N.

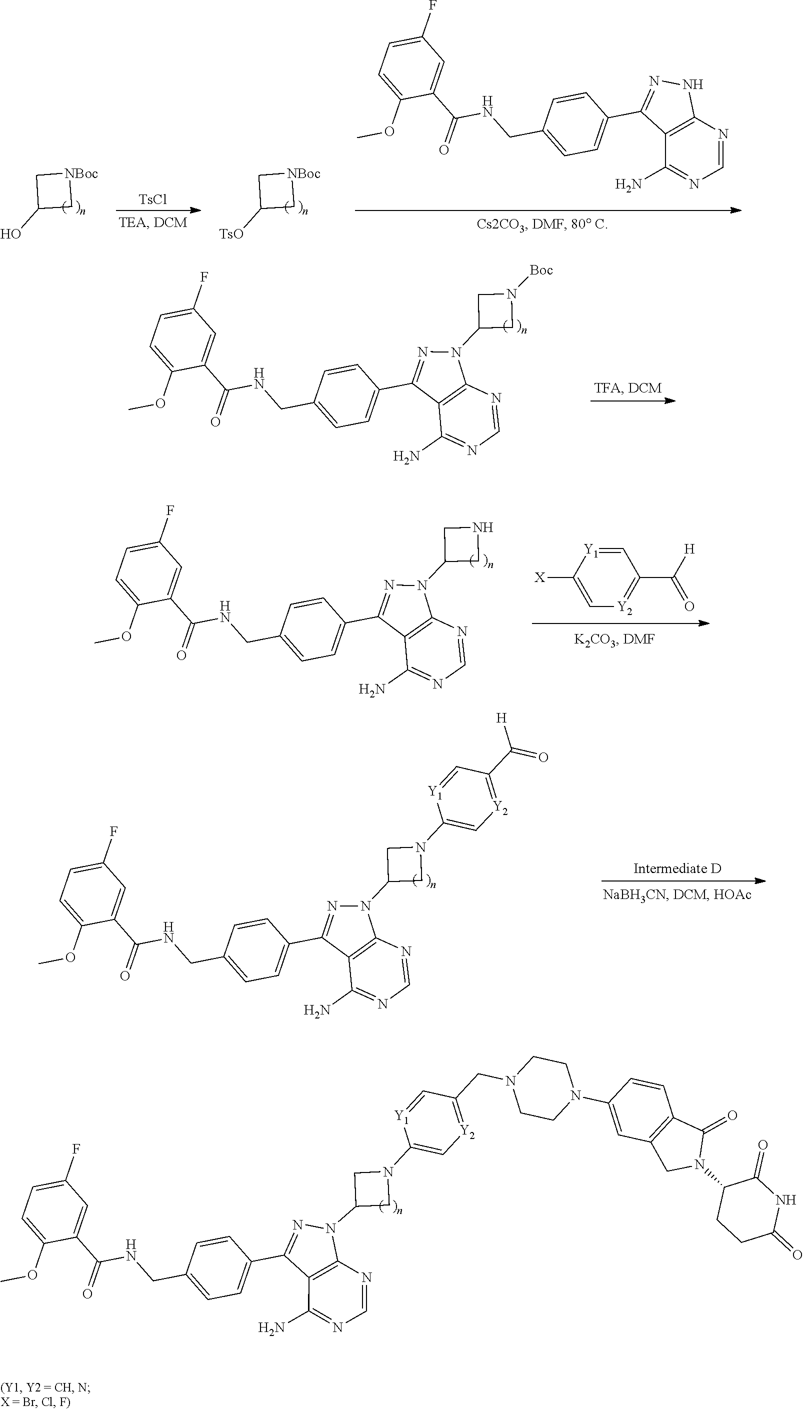

Scheme 3: Synthesis of BTK degrader molecules with Formula I-O.

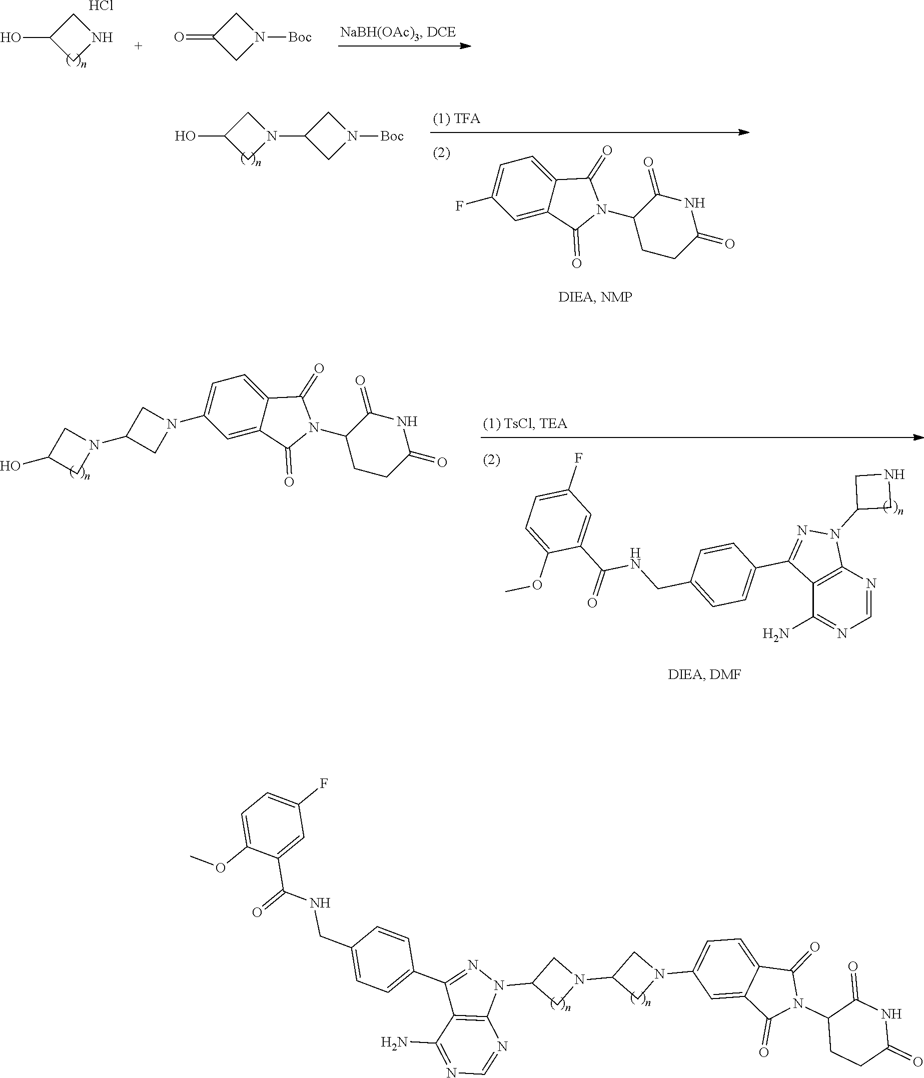

Scheme 4: Synthesis of BTK degrader molecules with Formula I-P.

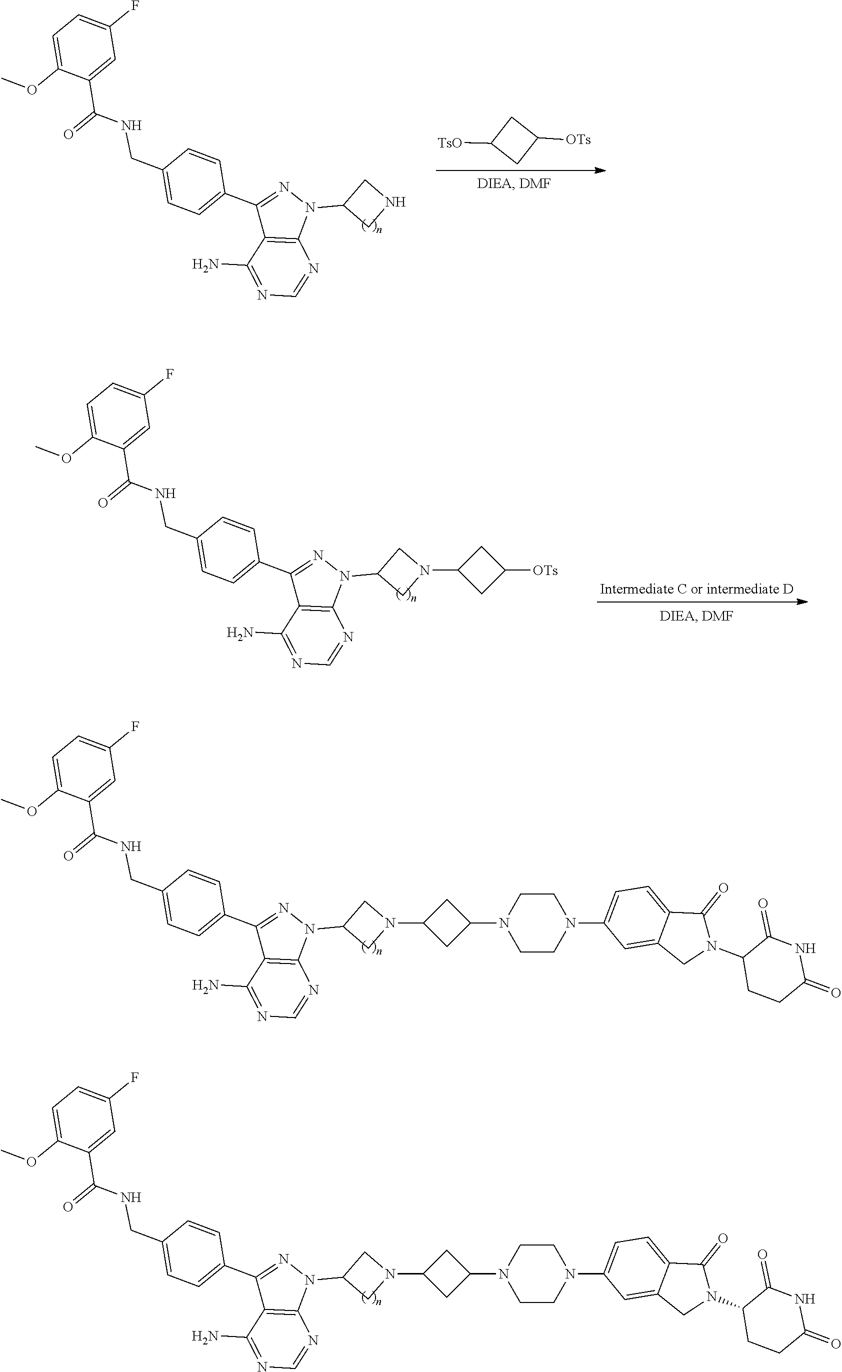 [SMILES: COc1ccc(F)cc1C(=O)NCc1ccc(-c2nn(C3CNC3)c3ncnc(N)c23)cc1 ; Cc1ccc(S(=O)(=O)OC2CC(OS(=O)(=O)c3ccc(C)cc3)C2)cc1 ; COc1ccc(F)cc1C(=O)NCc1ccc(-c2nn(C3CN(C4CC(OS(=O)(=O)c5ccc(C)cc5)C4)C3)c3ncnc(N)c23)cc1 ; COc1ccc(F)cc1C(=O)NCc1ccc(-c2nn(C3CN(C4CC(N5CCN(c6ccc7c(c6)CN(C6CCC(=O)NC6=O)C7=O)CC5)C4)C3)c3ncnc(N)c23)cc1 ; COc1ccc(F)cc1C(=O)NCc1ccc(-c2nn(C3CN(C4CC(N5CCN(c6ccc7c(c6)CN([C@H]6CCC(=O)NC6=O)C7=O)CC5)C4)C3)c3ncnc(N)c23)cc1]

BTK degrader molecules with Formula II-A, II-B and II-C can be prepared according to the methods described in WO 2021/113557, which is hereby incorporated by reference. BTK degrader molecules with Formula III-A, III-B, III-C and III-D can be prepared according to the methods described in WO 2020/239103 and WO 2022/052950, which are hereby incorporated by reference. BTK degrader molecules with Formula IV can be prepared according to the procedures described in WO 2021/219071, which is hereby incorporated by reference.

b. BTK Nucleic Acid Inhibitor Molecule

In another aspect, the BTK reducing molecule of the disclosure is a nucleic acid inhibitor molecule that reduces or eliminates the expression of BTK, leading to a reduction or elimination of the amount of BTK. As used herein, the term “nucleic acid inhibitor molecule” refers to an oligonucleotide molecule that reduces or eliminates the expression of a target gene wherein the oligonucleotide molecule contains a region that specifically targets a sequence in the target gene mRNA (i.e., BTK mRNA). Typically, the targeting region of the nucleic acid inhibitor molecule comprises a sequence that is sufficiently complementary to a segment of the target nucleic acid sequence (e.g., SEQ ID NO: 3) to direct the effect of the nucleic acid inhibitor molecule to the specified target gene (i.e., the BTK gene). The nucleic acid inhibitor molecule may include ribonucleotides, deoxyribonucleotides, and/or modified nucleotides. RNAi inhibitor molecules (including small interfering RNA (“siRNA”)), antisense oligonucleotides, ribozymes, microRNA, antagomirs, and aptamers are all examples of nucleic acid inhibitor molecules that have demonstrated capability to modulate intracellular RNA levels of a target gene. Antisense oligonucleotides and RNAi molecules against human BTK genes have been previously disclosed in, for example, U.S. Pat. No. 9,982,265, which is incorporated by reference herein in its entirety.

The sequence corresponding to the mRNA transcript of human BTK is shown below (SEQ ID NO: 3). This corresponds to the database sequence under GenBank accession No. NM_000061.3. a 2575-nucleotide sequence defined as “Homo sapiens Bruton tyrosine kinase (BTK), transcript variant 1, mRNA.” The coding region is located at nucleotides 161-2140. As is understood in the art, the thymine bases in SEQ ID NO: 3 are replaced with uracil bases in an RNA molecule. Thus, as used herein, a DNA sequence (such as SEQ ID NO: 3) and an RNA sequence corresponding to the DNA sequence (i.e., SEQ ID NO: 3 in which the thymine bases are replaced by uracil bases) are considered to contain the same “sequence.”

(SEQ ID NO: 3)
agactgtccttcctctctggactgtaagaatatgtctccagggccagt
gtctgctgcgatcgagtcccaccttccaagtcctggcatctcaatgca
tctgggaagctacctgcattaagtcaggactgagcacacaggtgaact
ccagaaagaagaagctatggccgcagtgattctggagagcatctttct
gaagcgatcccaacagaaaaagaaaacatcacctctaaacttcaagaa
gcgcctgtttctcttgaccgtgcacaaactctcctactatgagtatga
ctttgaacgtgggagaagaggcagtaagaagggttcaatagatgttga
gaagatcacttgtgttgaaacagtggttcctgaaaaaaatcctcctcc
agaaagacagattccgagaagaggtgaagagtccagtgaaatggagca
aatttcaatcattgaaaggttcccttatcccttccaggttgtatatga
tgaagggcctctctacgtcttctccccaactgaagaactaaggaagcg
gtggattcaccagctcaaaaacgtaatccggtacaacagtgatctggt
tcagaaatatcacccttgcttctggatcgatgggcagtatctctgctg
ctctcagacagccaaaaatgctatgggctgccaaattttggagaacag
gaatggaagcttaaaacctgggagttctcaccggaagacaaaaaagcc
tcttcccccaacgcctgaggaggaccagatcttgaaaaagccactacc
gcctgagccagcagcagcaccagtctccacaagtgagctgaaaaaggt
tgtggccctttatgattacatgccaatgaatgcaaatgatctacagct
gcggaagggtgatgaatattttatcttggaggaaagcaacttaccatg
gtggagagcacgagataaaaatgggcaggaaggctacattcctagtaa
ctatgtcactgaagcagaagactccatagaaatgtatgagtggtattc
caaacacatgactcggagtcaggctgagcaactgctaaagcaagaggg
gaaagaaggaggtttcattgtcagagactccagcaaagctggcaaata
tacagtgtctgtgtttgctaaatccacaggggaccctcaaggggtgat
acgtcattatgttgtgtgttccacacctcagagccagtattacctggc
tgagaagcaccttttcagcaccatccctgagctcattaactaccatca
gcacaactctgcaggactcatatccaggctcaaatatccagtgtctca
acaaaacaagaatgcaccttccactgcaggcctgggatacggatcatg
ggaaattgatccaaaggacctgaccttcttgaaggagctggggactgg
acaatttggggtagtgaagtatgggaaatggagaggccagtacgacgt
ggccatcaagatgatcaaagaaggctccatgtctgaagatgaattcat
tgaagaagccaaagtcatgatgaatctttcccatgagaagctggtgca
gttgtatggcgtctgcaccaagcagcgccccatcttcatcatcactga
gtacatggccaatggctgcctcctgaactacctgagggagatgcgcca
ccgcttccagactcagcagctgctagagatgtgcaaggatgtctgtga
agccatggaatacctggagtcaaagcagttccttcaccgagacctggc
agctcgaaactgtttggtaaacgatcaaggagttgttaaagtatctga
tttcggcctgtccaggtatgtcctggatgatgaatacacaagctcagt
aggctccaaatttccagtccggtggtccccaccggaagtcctgatgta
tagcaagttcagcagcaaatctgacatttgggcttttggggttttgat
gtgggaaatttactccctggggaagatgccatatgagagatttactaa
cagtgagactgctgaacacattgcccaaggcctacgtctctacaggcc
tcatctggcttcagagaaggtatataccatcatgtacagttgctggca
tgagaaagcagatgagcgtcccactttcaaaattcttctgagcaatat
tctagatgtcatggatgaagaatcctgagctcgccaataagcttcttg
gttctacttctcttctccacaagccccaatttcactttctcagaggaa
atcccaagcttaggagccctggagcctttgtgctcccactcaatacaa
aaaggcccctctctacatctgggaatgcacctcttctttgattccctg
ggatagtggcttctgagcaaaggccaagaaattattgtgcctgaaatt
tcccgagagaattaagacagactgaatttgcgatgaaaatatttttta
ggagggaggatgtaaatagccgcacaaaggggtccaacagctctttga
gtaggcatttggtagagcttgggggtgtgtgtgtgggggtggaccgaa
tttggcaagaatgaaatggtgtcataaagatgggaggggagggtgttt
tgataaaataaaattactagaaagcttgaaa.

In certain embodiments, the nucleic acid inhibitor molecule is a single-stranded nucleic acid inhibitor molecule. Single stranded nucleic acid inhibitor molecules include, for example, antisense oligonucleotides, microRNA, ribozymes, aptamers, antagomirs, and single stranded RNAi inhibitor molecules, all of which are known in the art.

As used herein, the term “antisense oligonucleotide” refers to single stranded oligonucleotides that inhibit the expression of a targeted gene by one of the following mechanisms: (1) steric hindrance, e.g., the antisense oligonucleotide interferes with some steps in the sequence of events involved in gene expression and/or production of the encoded protein by directly interfering with, for example, transcription of the gene, splicing of the pre-mRNA and translation of the mRNA; (2) induction of enzymatic digestion of the RNA transcripts of the targeted gene by RNase H; (3) induction of enzymatic digestion of the RNA transcripts of the targeted gene by RNase L; (4) induction of enzymatic digestion of the RNA transcripts of the targeted gene by RNase P: (5) induction of enzymatic digestion of the RNA transcripts of the targeted gene by double stranded RNase; and (6) combined steric hindrance and induction of enzymatic digestion activity in the same antisense oligo. Conventional antisense oligonucleotides do not have an RNAi mechanism of action like RNAi molecules. Antisense oligonucleotides have been used for decades to reduce expression of specific target genes. See e.g., Pelechano and Steinmetz, Nature Review Genetics, 2013, 14:880-93. RNAi molecules can be distinguished from antisense oligonucleotides in several ways including the requirement for Ago2 that combines with an RNAi antisense strand such that the antisense strand directs the Ago2 protein to the intended target(s) and where Ago2 is required for silencing of the target. An antisense oligonucleotide of the disclosure may be of any length that is effective for inhibition of the BTK gene/coding sequence.

Typically, an antisense oligonucleotide is between about 6 and about 50 nucleotides (e.g., at least about 12, 15, 20, 25, 30, 35, 40, 45 or 50 nucleotides), and may be as long as about 100 to about 200 nucleotides or more. In certain embodiments, the antisense oligonucleotide has 8-80, 14-50, 16-30, 12-25, 12-22, 14-20, 18-22, or 20-22 nucleotides. In certain embodiments, the antisense oligonucleotide has 18-22, such as 18-20 nucleotides. In certain embodiments, the antisense oligonucleotide or a portion thereof is fully complementary to a target nucleic acid sequence within SEQ ID NO: 3. In certain embodiments, the antisense oligonucleotide or a portion thereof is complementary to at least 12, 13, 14, 15, 16, 17, 18, 19, 20, or more contiguous nucleotides of the target nucleic acid sequence within SEQ ID NO: 3. In certain embodiments, the antisense oligonucleotide contains no more than 5, 4, 3, 2, or 1 non-complementary nucleotides relative to the target nucleic acid sequence within SEQ ID NO: 3. It is possible to decrease the length of the antisense oligonucleotide and/or introduce mismatch bases without eliminating activity.

In some embodiments, the nucleic acid inhibitor molecule is an antisense oligonucleotide comprising a single stranded polynucleotide comprising a sequence that is at least about 90% or at least about 91%, 92%, 93%, 94%, 95%, 96%, 97%, 98% or 99% complementary to a segment of the human BTK mRNA or coding DNA sequence (SEQ ID NO: 3).

In some embodiments, the nucleic acid inhibitor molecule is a RNAi molecule. As used herein, the term “RNAi molecule” refers to either (a) a double stranded nucleic acid inhibitor molecule (“dsRNAi molecule” also referred to in the art as siRNA molecules) having a sense strand (passenger) and antisense strand (guide), where the antisense strand or part of the antisense strand is used by the Argonaute 2 (Ago2) endonuclease in the cleavage of a target mRNA or (b) a single stranded nucleic acid inhibitor molecule (“ssRNAi molecule”) having a single antisense strand, where that antisense strand (or part of that antisense strand) is used by the Ago2 endonuclease in the cleavage of a target mRNA. See, e.g., Matsui et al., Molecular Therapy, 2016, 24(5):946-55.

In certain embodiments, the nucleic acid inhibitor molecule is a ssRNAi molecule. In certain embodiments, the nucleic acid inhibitor molecule is a ssRNAi molecule having 14-50, 16-30, or 15-25 nucleotides. In other embodiments, the ssRNAi molecule has 18-22 or 20-22 nucleotides. In certain embodiments, the ssRNAi molecule has 20 nucleotides. In other embodiments, the ssRNAi molecule has 22 nucleotides. In some embodiments, the ssRNAi molecule comprises a single-stranded polynucleotide comprising a sequence that is at least about 90% or at least about 91%, 92%, 93%, 94%, 95%, 96%, 97%, 98% or 99% complementary to a segment of the human BTK mRNA or coding DNA sequence (SEQ ID NO: 3).

In certain embodiments, the nucleic acid inhibitor molecule is a dsRNAi molecule. A variety of double stranded RNAi inhibitor molecule structures are known in the art. Early work on RNAi inhibitor molecules focused on double-stranded nucleic acid molecules with each strand having sizes of 19-25 nucleotides with at least one 3′-overhang of 1 to 5 nucleotides (see, e.g., U.S. Pat. No. 8,372,968). Subsequently, longer double-stranded RNAi inhibitor molecules that get processed in vivo by the Dicer enzyme to active RNAi inhibitor molecules were developed (see, e.g., U.S. Pat. No. 8,883,996). Later work developed extended double-stranded nucleic acid inhibitor molecules where at least one end of at least one strand is extended beyond the double-stranded targeting region of the molecule.

dsRNAi molecules of the disclosure may be of any length that is effective for inhibition of the BTK gene/coding sequence. In some embodiments of the dsRNAi molecule, the sense and antisense strands range from 15-66, 25-40, or 19-25 nucleotides. In some embodiments, the sense strand is between 18 and 66 nucleotides in length. In certain embodiments, the sense strand is between 18 and 25 nucleotides in length. In certain embodiments, the sense strand is 18, 19, 20, 21, 22, 23, or 24 nucleotides in length. In certain of those embodiments, the sense strand is between 25 and 45 nucleotides in length. In certain embodiments, the sense strand is between 30 and 40 nucleotides in length. In certain embodiments, the sense strand is 36, 37, 38, 39, or 40 nucleotides in length. In certain embodiments, the sense strand is between 25 and 30 nucleotides in length. In certain of those embodiments, the sense strand is 25, 26, or 27 nucleotides in length.

In some embodiments of the dsRNAi molecule, the antisense strand is between 18 and 66 nucleotides in length. Typically, the antisense strand comprises a sequence that is sufficiently complementary to a sequence in the BTK gene/coding sequence to direct the effect of the nucleic acid inhibitor molecule to the target BTK gene. In some embodiments, the antisense strand comprises a nucleic acid sequence that is at least about 90% or at least about 91%, 92%, 93%, 94%, 95%, 96%, 97%, 98% or 99% complementary to a segment of the human BTK mRNA or coding DNA sequence (SEQ ID NO: 3). In certain embodiments, the antisense strand comprises a sequence that is fully complementary with a sequence contained in the human BTK mRNA or coding DNA sequence (SEQ ID NO: 3).

In certain embodiments, the antisense strand is between 18 and 40 nucleotides long. In certain of those embodiments, the antisense strand is between 20 and 50 nucleotides in length. In certain embodiments, the antisense strand is between 20 and 30 nucleotides in length. In certain embodiments, the antisense strand is 21, 22, 23, 24, 25, 26, 27, or 28 nucleotides in length. In certain embodiments, the antisense strand is between 35 and 40 nucleotides in length. In certain of those embodiments, the antisense strand is 36, 37, 38, or 39 nucleotides in length.

In some embodiments of the dsRNAi inhibitor molecule, the sense and antisense strands form a duplex structure of between 15 and 50 base pairs. In certain embodiments, the duplex region is between 15 and 30 base pairs in length, such as between 19 and 30, more typically between 18 and 26, such as between 19 and 23, and in certain instances between 19 and 21 base pairs in length. In certain embodiments, the double-stranded region is 19, 20, 21, 22, 23, 24, 25, or 26 base pairs in length.

In certain embodiments, the dsRNAi inhibitor molecule comprises a sense and an antisense strand and a duplex region of between 19-21 nucleotides, wherein the sense strand is 19-21 nucleotides in length and the antisense strand is 21-23 nucleotides in length and comprises a single-stranded overhang of 1-2 nucleotides at its 3′-terminus.

In certain embodiments, the dsRNAi inhibitor molecule has an antisense strand of 21 nucleotides in length and a sense strand of 21 nucleotides in length, where there is a two nucleotide 3′-passenger strand overhang on the right side of the molecule (3′-end of sense strand/5′-end of antisense strand) and a two nucleotide 3′-guide strand overhang on the left side of the molecule (5′-end of the sense strand/3′-end of the antisense strand). In such molecules, there is a 19 base pair duplex region.

In certain embodiments, the dsRNAi inhibitor molecule has an antisense strand of 23 nucleotides in length and a sense strand of 21 nucleotides in length, where there is a blunt end on the right side of the molecule (3′-end of sense strand/5′-end of antisense strand) and a two nucleotide 3′-guide strand overhang on the left side of the molecule (5′-end of the sense strand/3′-end of the antisense strand). In such molecules, there is a 21 base pair duplex region.

In some embodiments, the nucleic acid inhibitor molecule is a microRNA. As used herein, the terms “microRNA” and “miRNA” are interchangeable and refer to non-coding RNA molecules encoded in the genomes of plants and animals. Typically, mature microRNA are about 18-25 nucleotides in length. In certain instances, highly conserved, endogenously expressed microRNAs regulate the expression of genes by binding to the 3′-untranslated regions (3′-UTR) of specific mRNAs. Certain mature microRNAs appear to originate from long endogenous primary microRNA transcripts (also known as pre-microRNAs, pri-microRNAs, pri-mirs, pri-miRs or pri-pre-microRNAs) that are often hundreds of nucleotides in length (Lee, et al., EMBO J., 2002, 21(17):4663-4670).

In some embodiments, the nucleic acid inhibitor molecule is an aptamer. As used herein, the term “aptamer” refers to an oligonucleotide that has binding affinity for a specific target including a nucleic acid, a protein, a specific whole cell or a particular tissue. Aptamers may be obtained using methods known in the art, for example, by in vitro selection from a large random sequence pool of nucleic acids. Lee et al., Nucleic Acid Res., 2004, 32:D95-D100.

In some embodiments, the nucleic acid inhibitor molecule is an antagomir. As used herein, the term “antagomir” refers to an oligonucleotide that has binding affinity for a specific target including the guide strand of an exogenous RNAi inhibitor molecule or natural miRNA (Krutzfeldt et al. Nature 2005, 438(7068):685-689).

In some embodiments, the nucleic acid inhibitor molecule is a ribozyme. As used herein, the term “ribozyme” refers to a catalytic nucleic acid molecule that specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

Methods of making nucleic acid inhibitor molecules, such as antisense oligonucleotides or RNAi molecules, are conventional. For instance, in vitro methods for making RNAi molecules include processing the polyribonucleotide sequence in a cell-free system (e.g., digesting long dsRNAs with RNAse III or Dicer), transcribing recombinant double stranded DNA in vitro, and, preferably, chemical synthesis of nucleotide sequences homologous to BTK sequence. See, e.g., Tuschl et al., Genes & Dev., 1999, 13:3191-3197. In vivo methods, on the other hand, may include:
(1) transfecting DNA vectors into a cell such that a substrate is converted into RNAi molecule in vivo (see, for example, Kawasaki et al., Nucleic Acids Res., 2003, 31:700-707; Miyagishi et al., Nature Biotechnol., 2003, 20:497-500; Lee et al., Nature Biotechnol., 2003, 20:500-505; Brummelkamp et al., Science, 2003, 296:550-53; McManus et al., RNA, 2002, 8:842-850; Paddison et al., Gene. Dev., 2002, 16:948-958; Paddison et al., PNAS, 2002, 99:1443-1448; Paul et al., Nature Biotechnol., 2002, 20:505-508; Yu et al., PNAS, 2002, 99:6047-6052);
(2) expressing shRNAs from plasmid systems using RNA polymerase III (pol III) promoters (see, for example, Kawasaki et al., supra; Miyagishi et al., supra; Lee et al., supra; Brummelkamp et al., supra; McManus et al., supra; Paddison et al., supra (both); Paul et al., supra, and Yu et al., supra); and/or
(3) expressing short RNA from tandem promoters (see, for example, Miyagishi et al., supra; Lee et al., supra).

When synthesized in vitro, a typical micromolar scale RNA synthesis provides about 1 mg of RNAi molecule, which is sufficient for about 1000 transfection experiments using a 24-well tissue culture plate format. In general, to inhibit BTK expression in cells in culture, one or more RNAi molecules can be added to cells in culture media, typically at about 1 ng/ml to about 10 g RNAi molecule/ml.

For further guidance for methods of designing and preparing RNAi molecules, testing them for efficacy, and using them in methods of RNAi (both in vitro and in vivo), see, e.g., Allshire, Science, 2002, 297:1818-1819; Volpe et al., Science, 2002, 297:1833-1837; Jenuwein, Science, 2002, 297:2215-2218; Hall et al., Science, 2002, 297:2232-2237; Hutvagner et al., Science, 2002, 297:2056-2060; McManus et al., supra; Reinhart et al., Genes. Dev., 2002. 16:1616-1626; Reinhart et al., Science, 2002, 297:1831; Moss, Curr. Biol., 2001, 11:R772-775; Brummelkamp et al., Science, 2002, 296:550-553; Bass, Nature, 2001, 411:428-429; Elbashir et al., supra; U.S. Pat. No. 6,506,559; US Pat Appl. 2003/0206887; WO99/07409; WO99/32619; WO00/01846; WO 00/44914; WO00/44895; WO2001/29058; WO2001/36646; WO2001/75164; WO2001/92513; WO2001/29058; WO2001/89304; WO2001/90401; WO2002/16620; and WO2002/29858, all of which are incorporated herein by reference.

The nucleic acid inhibitor molecules of the disclosure, such antisense oligonucleotides, RNAi molecules, microRNA, aptamers, antagomirs, or ribozymes, can take any of the forms, including modified versions, described for antisense nucleic acid molecules; and they can be delivered to cells and introduced into cells as oligonucleotides (single or double stranded) or in the form of an expression vector using any methods known in the art.

### Dosage Forms and Compositions

The methods disclosed herein comprise administering to a subject an effective amount of a BTK reducing molecule. This may occur subsequent to having identified the subject as having a disease or disorder associated with a constitutively activated PLCγ2, such as a cancer or an immune system disorder, by, for example, identifying the subject as having constitutively activated PLCγ2 or one or more gain-of-function mutations in the gene encoding PLCγ2 in one or more cells.

Accordingly, the disclosure provides pharmaceutical compositions that contain, as the active ingredient, a BTK reducing molecule disclosed herein and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modem Pharmaceutics, Marcel Dekker, Inc. 3rd Ed. (G. S. Banker & C. T. Rhodes, Eds.).

The pharmaceutical compositions may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The pharmaceutical compositions may be administered in either single or multiple doses by any of the accepted modes of administration of agents having similar utilities, for example as described in those patents and patent applications incorporated by reference, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. In some embodiments, the compounds or pharmaceutical compositions of the disclosure are administered orally.

One mode for administration is parenteral, particularly by injection. The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of BTK reducing molecules in accordance with the present disclosure. Administration may be via capsule or tablets, or the like. In making the pharmaceutical compositions that include at least one BTK reducing molecule described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the disclosure can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form of about 1-1000 mg of active ingredient(s) for a subject of about 50-70 kg, or about 1-500 mg or about 1-250 mg or about 1-150 mg or about 0.5-100 mg, or about 1-50 mg of active ingredients. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The BTK reducing molecules are generally administered in a pharmaceutically effective amount. Preferably, for oral administration, each dosage unit contains from about 1 mg to about 2 g of a BTK reducing molecule described herein, and for parenteral administration, preferably from about 0.1 to about 700 mg of a BTK reducing molecule described herein. It will be understood, however, that the amount of the BTK reducing molecule actually administered usually will be determined by a physician, in the light of the relevant circumstances, including the condition to be treated, the chosen route of administration, the actual BTK reducing molecule administered and its relative activity, the age, weight, and response of the individual patient, the severity of the patient's symptoms, and the like.

In some embodiments, the BTK reducing molecule of the disclosure, such as any of the BTK degrader molecules disclosed herein, is administered to the subject at a dose of from about 0.1 mg/kg to about 500 mg/kg (e.g., from about 0.5 mg/kg to about 400 mg/kg, from about 0.7 mg/kg to about 300 mg/kg, from about 1 mg/kg to about 250 mg/kg, from about 1.5 mg/kg to about 200 mg/kg, from about 2 mg/kg to about 150 mg/kg, from about 1 mg/kg to about 100 mg/kg, from about 0.1 mg/kg to about 100 mg/kg, from about 0.1 mg/kg to about 50 mg/kg, or from about 0.1 mg/kg to about 10 mg/kg).

For preparing solid compositions such as tablets, the principal active ingredient is mixed with a pharmaceutical excipient to form a solid preformulation composition containing a homogeneous mixture of a BTK reducing molecule of the present disclosure. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In one aspect, provided herein is a dosage form or a composition in a dosage form comprising: from about 0.1 mg to about 1 g (e.g., from about 0.1 mg to about 750 mg, from about 0.2 mg to about 500 mg, from about 0.5 mg to about 200 mg, from about 1 mg to about 150 mg, from about 2.5 mg to about 150 mg, from about 10 mg to about 120 mg) of a BTK reducing molecule disclosed herein and a pharmaceutically acceptable excipient.

In some embodiments, the dosage form or a composition in a dosage form comprises about 1 g, about 750 mg, about 500 mg, about 200 mg, 190 mg, 180 mg, 170 mg, 160 mg, 150 mg, 140 mg, 130 mg, 120 mg, 110 mg, 100 mg, about 90 mg, about 85 mg, about 80 mg, about 75 mg, about 70 mg, about 65 mg, about 60 mg, about 55 mg, about 50 mg, about 45 mg, about 40 mg, about 35 mg, about 30 mg, about 25 mg, about 20 mg, about 15 mg, about 10 mg, about 7 mg, about 5 mg, about 2.5 mg, about 2 mg, about 1.5 mg, or about 1 mg of a BTK reducing molecule disclosed herein.

In another aspect, the present disclosure provides a dosage form or a composition in a dosage form comprising a plurality of particles of a BTK reducing molecule disclosed herein and a pharmaceutically acceptable excipient, wherein the amount of the plurality of particles of the BTK reducing molecule disclosed herein in the dosage form is from about 0.1 mg to about 500 mg (e.g., from about 0.5 mg to about 200 mg, from about 1 mg to about 150 mg, from about 10 mg to about 120 mg).

In some embodiments, the plurality of particles of a BTK reducing molecule disclosed herein in the dosage form or composition is from about 2.5 mg to about 150 mg (e.g., from about 10 mg to about 150 mg, from about 20 mg to about 150 mg, from about 70 mg to about 120 mg, from about 30 mg to about 60 mg, about 100 mg, about 50 mg).

In some embodiments, the dosage form or the composition is configured for oral administration. In some embodiments, the dosage form is a solid form. In some embodiments, the dosage form is in the form of a capsule. In some embodiments, the pharmaceutical excipient in the capsule is a filler (e.g., cellulose derivatives (e.g., microcrystalline cellulose), starches (e.g., hydrolyzed starches, and partially pregelatinized starches), anhydrous lactose, lactose monohydrate, sugar alcohols (e.g., sorbitol, xylitol, and mannitol).

In some embodiments, the dosage form is a liquid form. In some embodiments, the dosage form is in the form of a solution. In some embodiments, the pharmaceutical excipient in the solution is selected from the group consisting of a filler (e.g., polymer (e.g., PEG 400)), an emulsifier (e.g., a castor oil derivative (e.g., Kolliphor RH40), a surfactant (e.g., a glyceride (e.g., Labrafil M2125 CS), a vitamin derivative (e.g., Vitamin ETPGS)), a solvent (e.g., propylene glycol, ethanol, diethylene glycol monoethyl ether (or Transcutol HP)).

In some embodiments, the concentration of a BTK reducing molecule disclosed herein in the solution is from about 0.1 mg/mL to about 10 mg/mL (e.g., from about 0.5 mg/mL to about 10 mg/mL, from about 1 mg/mL to about 10 mg/mL, from about 2 mg/mL to about 10 mg/mL, from about 3 mg/mL to about 10 mg/mL, from about 4 mg/mL to about 10 mg/mL, from about 5 mg/mL to about 10 mg/mL, from about 6 mg/mL to about 10 mg/mL, from about 0.1 mg/mL to about 8 mg/mL, from about 0.5 mg/mL to about 8 mg/mL, from about 1 mg/mL to about 8 mg/mL, from about 2 mg/mL to about 8 mg/mL, from about 3 mg/mL to about 8 mg/mL, from about 4 mg/mL to about 8 mg/mL, from about 5 mg/mL to about 8 mg/mL, from about 6 mg/mL to about 8 mg/mL, from about 0.5 mg/mL to about 6 mg/mL, from about 1 mg/mL to about 6 mg/mL, from about 2 mg/mL to about 6 mg/mL, from about 3 mg/mL to about 6 mg/mL, from about 4 mg/mL to about 6 mg/mL, from about 0.5 mg/mL to about 4 mg/mL, from about 1 mg/mL to about 4 mg/mL, or from about 2 mg/mL to about 4 mg/mL).

In some embodiments, the concentration of a BTK reducing molecule disclosed herein in the solution is about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL.

In some embodiments, the dosage form is in the form of a suspension. In some embodiments, the concentration of a BTK reducing molecule disclosed herein in the suspension is about 0.1 mg/mL, about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 20 mg/mL, about 25 mg/mL.

In some embodiments, the concentration of a BTK reducing molecule disclosed herein in the suspension is from about 0.1 mg/mL to about 10 mg/mL (e.g., from about 0.5 mg/mL to about 10 mg/mL, from about 1 mg/mL to about 10 mg/mL, from about 2 mg/mL to about 10 mg/mL, from about 3 mg/mL to about 10 mg/mL, from about 4 mg/mL to about 10 mg/mL, from about 5 mg/mL to about 10 mg/mL, from about 6 mg/mL to about 10 mg/mL, from about 0.1 mg/mL to about 8 mg/mL, from about 0.5 mg/mL to about 8 mg/mL, from about 1 mg/mL to about 8 mg/mL, from about 2 mg/mL to about 8 mg/mL, from about 3 mg/mL to about 8 mg/mL, from about 4 mg/mL to about 8 mg/mL, from about 5 mg/mL to about 8 mg/mL, from about 6 mg/mL to about 8 mg/mL, from about 0.5 mg/mL to about 6 mg/mL, from about 1 mg/mL to about 6 mg/mL, from about 2 mg/mL to about 6 mg/mL, from about 3 mg/mL to about 6 mg/mL, from about 4 mg/mL to about 6 mg/mL, from about 0.5 mg/mL to about 4 mg/mL, from about 1 mg/mL to about 4 mg/mL, or from about 2 mg/mL to about 4 mg/mL).

Administration in vivo can be affected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

### EXAMPLES

In order that the embodiments described herein may be more fully understood, the following examples are set forth. The examples described in this application are offered to illustrate the compounds, pharmaceutical compositions, and methods provided herein and are not to be construed in any way as limiting their scope.

### Example 1. Effect of BTK Reducing Molecule of Formula I-D and BTK Inhibitor Ibrutinib in Inhibiting Cancer Cell Growth In Vitro

Parental REC1 cells were obtained from the American Type Culture Collection (ATCC). PLCγ2 gene in REC-1 cell line was edited using the CRISPR-Cas9 technology to create a S to Y point mutation at residue 707 of PLCγ2 protein, or L to F point mutation at residue 845 of PLCγ2 protein. For gene editing, first, gRNA complex of PLCγ2 S707Y or PLCγ2 L845F was prepared with Alt-R CRISPR-Cas9 tracrRNA and crRNA PLCγ2 S707Y (5′-CAGACTCTCAAAATAGGCGG-3′) or PLCγ2 L845F (5′-TTATTGAAGACAATCCCTTA-3′) (IDT-Integrated DNA Technologies), and then ribonucleoprotein (RNP) complex of PLCγ2 S707Y or PLCγ2 L845F was prepared with respective gRNA complex and Alt-R S.p. Cas9 Nuclease V3 (IDT-Integrated DNA Technologies). Next, 1×10⁵ REC-1 cells were electroporated with PLCγ2 S707Y or PLCγ2 L845F RNP complex, Alt-R Cas9 Electroporation Enhancer and respective Ultramer DNA Oligo of PLCγ2 S707Y (5′-TCCTGCTCCAGGGCTAGGGGCAAGGTAAAGCATTGTCGCATCAACCGGGACGGCCG GCACTTTGTGCTGGGGACCTACGCTTATTTTGAGAGTCTGGTGGAGCTCGTCAGTTA CTACGAGAAGCATTCACTCTACCGAAAGATGAGACTGCGCT-3′) or PLCγ2 L845F (5′-TTTTCTTTTTATTATTCCCGTTACAACTAACGTGAGTTATGTCTTGTTTCTTCACAGAT TATTGAAGACAATCCCTTTGGCTCTCTTTGCAGAGGAATATTGGACCTCAATACCTA TAACGTCGGTACGTGCACACATCATCTTAGCCTGGAT-3′) (IDT-Integrated DNA Technologies) using Neon Transfection System (Thermo Fisher) under the condition of 1400 V, 10 ms, 3 pulses. Cells were then plated in a 96-well plate for 10 days and selected with 100 nM Ibrutinib for at least 6 passages. S707Y and L845F mutation of PLCγ2 was further validated by Sanger Sequencing using gDNA extracted from these cells.

The BTK reducing molecule used in this study was a BTK degrader having the chemical structure of Formula I-D shown below and described in PCT Application No. PCT/US22/14830, incorporated by reference herein, and was prepared according to the method described therein. The BTK inhibitor used in this study was Ibrutinib.

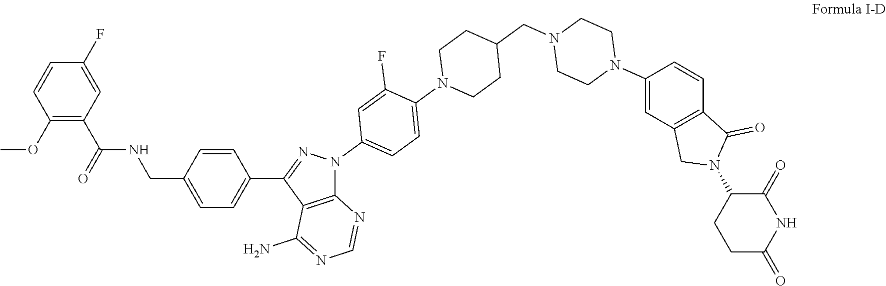

Parental REC1 cells (ATCC), engineered REC1 PLCγ2^{S707Y} cells, or engineered REC-1 PLCγ2^{L845F} cells, were plated in 96-well plates at 8,000 cells/well in 90 μl of RPMI1640 growth medium containing 10% heat-inactivated FBS and 1× Penicillin Streptomycin, and then incubated at 37° C. overnight. The following day, the test compound was administered to the cells by using 10× compound stock solution prepared in growth medium at various concentrations. After administration of the compound, cells were then incubated at 37° C. for 6 days. Before CellTiter-Glo assay, the plates were equilibrated at room temperature for approximately 10 minutes. 100 μl of CELLTITER-GLO® Reagent (Promega) was added to each well. The plates were then incubated at room temperature for 10 minutes and luminescence was recorded by EnSpire plate reader (PerkinElmer).

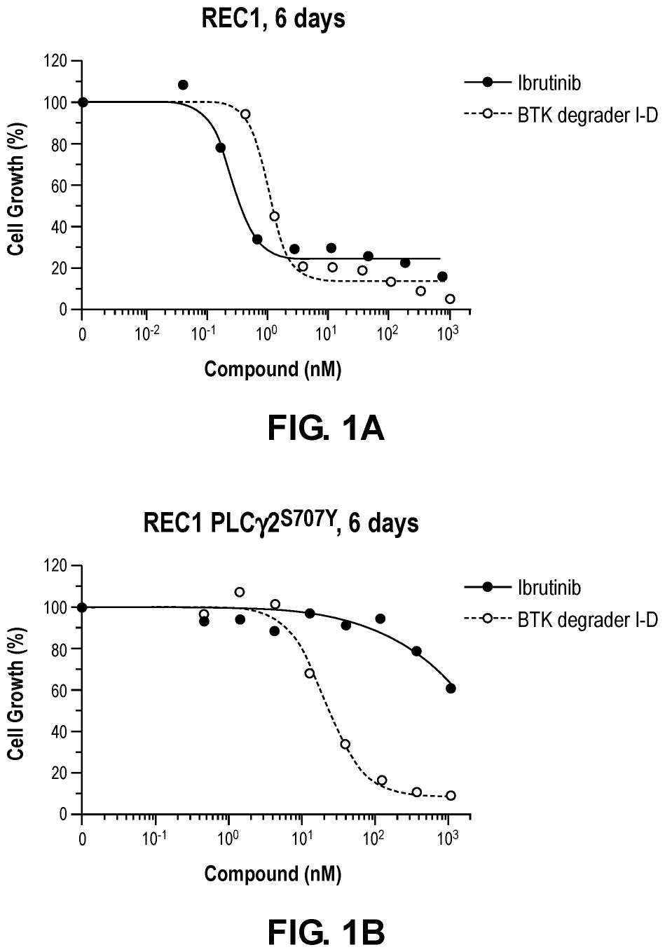

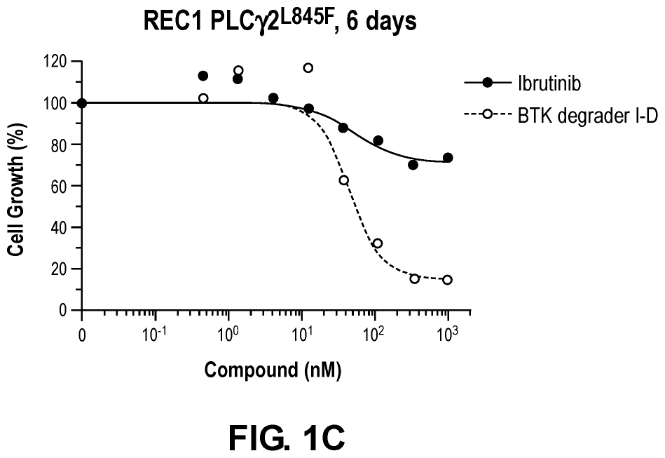

The results are shown in FIG. 1A-1C. As demonstrated in FIG. 1B and FIG. 1C, in vitro treatment with the BTK degrader of Formula I-D (“BTK degrader I-D”) significantly inhibited cell growth in engineered REC1 PLCγ2^{S707Y} cells and engineered REC-1 PLCγ2^{L845F} cells 6 days after administration, as compared to in vitro treatment with Ibrutinib, a conventional small molecule BTK inhibitor.

### Example 2. In Vivo Efficacy of BTK Reducing Molecule of Formula I-D and BTK Inhibitor Ibrutinib in a Mouse Xenograft Tumor Model

Parental REC1 cells (ATCC), engineered REC1 PLCγ2^{S707Y} cells, and engineered REC-1 PLCγ2^{L845F} cells, as described in Example 1, were maintained in vitro as monolayer culture in RPMI1640 medium supplemented with 10% fetal bovine serum and 1× Penicillin Streptomycin at 37° C. in an atmosphere of 5% CO2 in air. The tumor cells were routinely subcultured twice weekly. The cells growing in an exponential growth phase were harvested and counted for tumor inoculation. Each mouse was inoculated subcutaneously at the armpit of the right flank with 5×10⁶ tumor cells in 0.1 mL of PBS mixed with 0.1 mL matrigel for tumor development. Treatment of the BTK degrader of Formula I-D (1.5 mg/kg, 3 mg/kg, 6 mg/kg, or 15 mg/kg, PO), Ibrutinib (25 mg/kg, PO), or vehicle (PO) started when the average tumor size reached approximately 100 mm³. Mice were assigned into groups using an Excel-based randomization software performing stratified randomization based upon their tumor volumes. Tumor sizes were measured 3 times weekly in two dimensions using a caliper, and the volume was expressed in mm³ using the formula: V=0.5 a×b² where a and b are the long and short diameters of the tumor, respectively. Tumor growth inhibition (TGI) was calculated for each group using the formula: TGI (%)=[1−(Ti−T0)/(Vi−V0)]×100; Ti is the average tumor volume of a treatment group on a given day, T0 is the average tumor volume of the treatment group on the first day of treatment, Vi is the average tumor volume of the vehicle control group on the same day with Ti, and V0 is the average tumor volume of the vehicle group on the first day of treatment.

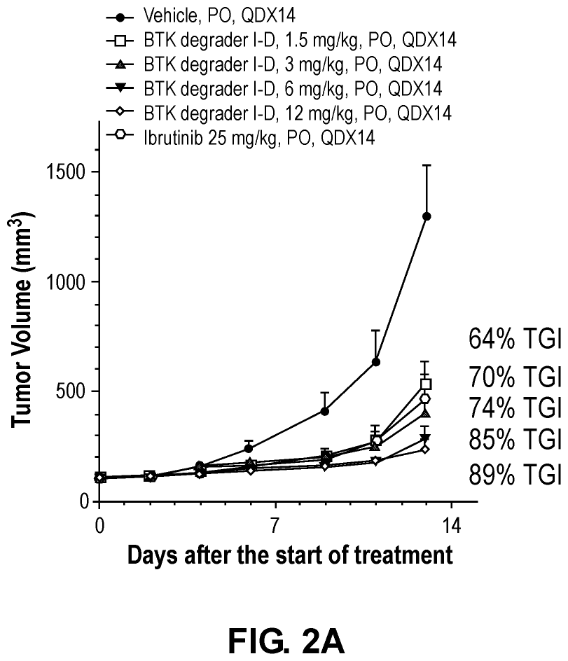

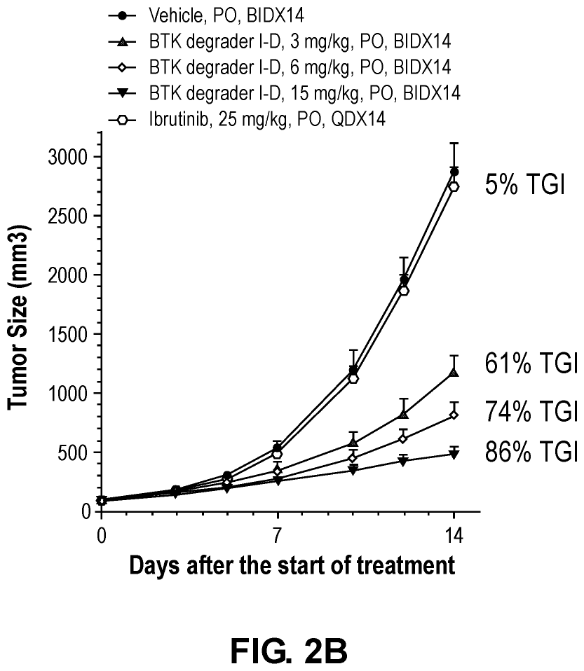

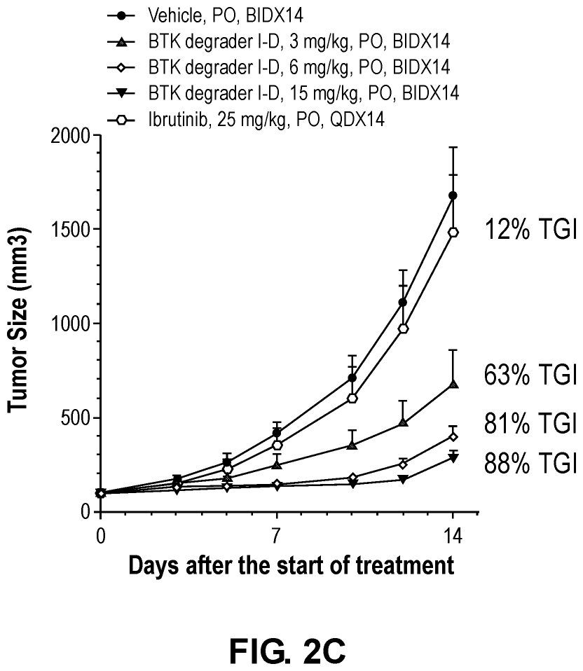

The results are shown in FIG. 2A-2C. FIG. 2A shows that Ibrutinib and BTK degrader of Formula I-D (“BTK degrader I-D”) have similar effect in inhibiting tumor growth in control mice without PLCγ2 mutation. In vivo treatment with the BTK degrader of Formula I-D, however, significantly inhibited tumor growth in engineered REC1 PLCγ2^{S707Y} knock-in mice and REC-1 PLCγ2^{L845F} knock-in mice 14 days after administration, as compared to in vivo treatment with Ibrutinib, as demonstrated in FIG. 2B and FIG. 2C. As shown in FIG. 2B, in vivo treatment of mice carrying engineered REC1 PLCγ2^{S707Y} knock-in tumors with the BTK degrader of Formula I-D at a dosage of 3 mg/kg, 6 mg/kg, or 15 mg/kg (PO) resulted in a TGI of 61%, 74% or 86%, respectively, 14 days after administration, while in vivo treatment with Ibrutinib only resulted in 5% TGI. Similarly, as shown FIG. 2C, in vivo treatment of mice carrying engineered REC1 PLCγ2^{L845F} knock-in tumors with the BTK degrader of Formula I-D at a dosage of 3 mg/kg, 6 mg/kg, or 15 mg/kg (PO) resulted in a TGI of 63%, 81% or 88%, respectively, 14 days after administration, while in vivo treatment with Ibrutinib only resulted in 12% TGI.

### Example 3. Analysis of Inositol Phosphate Formation in COS-7 Cells Transfected with Wild-Type and Mutant PLCγ2

The activity of PLCγ2 may be determined by analyzing the inositol phosphate formation in cells, such as COS-7 cells, transfected with wild-type or mutant PLCγ2 as described in Everett et al., Characterization of Phospholipase Cγ Enzymes with Gain-of-Function Mutations, J. Biol. Chem., 2009, 284(34):23083-23093.

Briefly, COS-7 cells are maintained in Dulbecco's modified Eagle's medium (DMEM) supplemented with 10% fetal bovine serum and 1× Penicillin Streptomycin at 37° C. in an atmosphere of 5% CO2 in air. Prior to transfection, COS-7 cells are seeded into 6-well plates at a density of 250,000 cells/well and grown overnight in 2 ml growth media before transfection. 1 μg plasmid DNA of wild-type PLCγ2 or mutant PLCγ2 is transfected using Lipofectamine (Invitrogen) according to the manufacture's protocol. 24 hours after transfection, COS-7 cells are washed twice with inositol-free DMEM without serum and incubated for 24 hours in 1.5 ml of the same medium supplemented with 0.25% fatty acid free bovine serum albumin (Sigma) and 1.5 uCi/ml myo-[2-³H] inositol (MP Biomedicals). After a further 24 hours, the cells are incubated in 1.2 ml of inositol-free DMEM without serum containing 20 mM LiCl with or without stimulation with 100 ng/ml EGF (Calbiochem) for 1 hour.

The cells are lysed by addition of 1.2 ml of 4.5% perchloric acid and incubated on ice for 30 minutes. Lysed samples are then centrifuged for 20 minutes at 3700 g. The supernatants are taken out and neutralized by addition of 3 ml of 0.5 M potassium hydroxide/9 mM sodium tetraborate and centrifuged for a further 20 minutes at 3700 g.

Supernatants are loaded onto AG1-X8 200-400 columns (Bio-Rad) that is converted to the formate form by addition of 2 M ammonium formate/0.1 M formic acid and equilibrated with water. The columns are washed three times with 5 ml of 60 mM ammonium formate/5 mM sodium tetraborate, and inositol phosphates are eluted with 5 ml of 1.2 M ammonium formate/0.1 M formic acid. 5 ml Ultima-Flo scintillation fluid (PerkinElmer Life Sciences) is added to the eluates and the radioactivity quantified by liquid scintillation counting. The values represent total inositol phosphates.

The pellets from the first centrifugation are resuspended in 100 μl of water and 375 μl of chloroform/methanol/HCl (200:100:15) is added. The samples are vortexed, and an additional 125 μl of chloroform and 125 μl of 0.1 M HCl are added. After further vortexing, the samples are centrifuged at 700 g for 10 minutes. 10 μl of the lower phase are placed in a scintillation vial with 3 ml of Ultima-Flo scintillation fluid and the radioactivity quantified by liquid scintillation counting. The obtained values correspond to radioactivity in inositol lipids.

PLCγ2 activity is expressed as the total [3H]-inositol phosphates formed relative to the amount of [³H]myo-inositol in the phospholipid pool. PLCγ2 mutants that show higher activity than wild-type PLCγ2 in the above assay are considered as gain-of-function mutants.

### Example 4. Assays for Detecting the Activity of PLCγ2 in DT40 Cells Stably Expressing Either Wild-Type or Mutated PLCγ2

The activity of PLCγ2 may also be determined by measuring the increased level of calcium flux in DT40 cells stably expressing either wild-type or mutated PLCγ2 as described in Woyach et al., Resistance Mechanisms for the Bruton's Tyrosine Kinase Inhibitor Ibrutinib, New England Journal of Medicine, 2014, 370:2286-2294.

Briefly, the intracellular calcium level of DT40 cells stably expressing either wild-type or mutated PLCγ2 is detected by Calcium Assay Kit (BD Biosciences) and measured by Beckman Coulter DTX880 microplate reader following the manufactures' protocols. After 195 seconds of acquisition to determine the baseline, 3 g/ml anti-chicken IgM (SouthernBiotech) is added to stimulate the cells and the fluorescent signal is recorded for additional 660 seconds.

PLCγ2 mutants that show higher calcium influx signal than wild-type PLCγ2 in the above assay are considered as gain-of-function mutants.

### Example 5. Assays for Detecting the Activity of PLCγ2 in Primary Patient Cells

The activity of PLCγ2 can also be determined by measuring the increased level of calcium flux stimulated by IP3 as described in Novice et al., A Germline Mutation in the C2 Domain of PLCγ2 Associated with Gain-of-Function Expands the Phenotype for PLCG2-Related Diseases, Journal of Clinical Immunology, 2020, 40:267-276.

Briefly, PBMCs from a subject (e.g., patient or healthy control) are washed once in HBSS (no Ca²⁺, no Mg²⁺, Life Technologies)+1% FBS (GIBCO®) and resuspended in dye-loading buffer consisting of 4 μM FLUO-4AM (Molecular Probes) and Probenecid (Life Technologies) in HBSS+1% FBS for 45 minutes at 37° C. at a concentration of 1×10⁶ PBMCs/mL. Cells are washed again with HBSS+1% FBS and then incubated on ice for 20 minutes with 5 μL of PACIFIC BLUE™-CD19 (HIB19; BioLegend) followed by addition of 1 mL HBSS+1% FBS. Samples are warmed again to 37° C. and within 10 minutes baseline fluorescence is detected with FITC filter on LSR II flow cytometer in the CD19+ positive fraction. Intracellular calcium flux is induced by B cell receptor stimulation with 10 μg/mL anti-IgM antibody (Jackson Immunoresearch) followed by the addition of extracellular Ca²⁺ to measure external flux. Intracellular and plasma calcium flux after B cell receptor stimulation in the subject's primary B cells are measured by flow cytometry.

An increase in external calcium entry in the subject's primary B cells induced by B cell receptor stimulation relative to controls indicates that the subject has a gain-of function mutation in PLCγ2

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. In general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

We claim:

1. A method of treating a disease or disorder associated with a constitutively activated phosphatidylinositol-specific phospholipase Cγ2 (PLCγ2) in a subject in need thereof, the method comprising administering to the subject an effective amount of a Bruton's tyrosine kinase (BTK) reducing molecule.

2. A method of treating a disease or disorder associated with a constitutively activated phosphatidylinositol-specific phospholipase Cγ2 (PLCγ2) in a subject in need thereof, the method comprising administering to the subject an effective amount of a Bruton's tyrosine kinase (BTK) reducing molecule, wherein, prior to administering to the subject the BTK reducing molecule, the subject has been identified as having constitutively activated PLCγ2 in one or more cells.

3. The method of claim 1 or 2, wherein the constitutively activated PLCγ2 is caused by one or more gain-of-function mutations in a gene encoding PLCγ2.

4. The method of any one of claims 1-3, wherein the disease or disorder associated with the constitutively activated PLCγ2 is a cancer.

5. The method of claim 4, wherein the cancer is a hematological cancer or a solid tumor.

6. The method of claim 5, wherein the hematological cancer is a B-cell malignancy.

7. The method of claim 6, wherein the B-cell malignancy is non-Hodgkin lymphoma (NHL).

8. The method of claim 7, wherein the non-Hodgkin lymphoma (NHL) is chronic lymphocytic leukemia (CLL), small lymphocytic lymphoma (SLL), mantle cell lymphoma (MCL), marginal zone lymphoma (MZL), or Waldenstrom macroglobulinemia (WM).

9. The method of any one of claims 4-8, wherein the cancer is resistant to a BTK inhibitor.

10. The method of claim 9, wherein the BTK inhibitor is ibrutinib, acalabrutinib, zanubrutinib, or tirabrutinib.

11. The method of any one of claims 4-10, wherein the method further comprises identifying a cancer cell obtained from the subject as having the constitutively activated PLCγ2 relative to a non-cancer cell obtained from the subject.

12. The method of any one of claims 4-11, wherein the method further comprises identifying a cancer cell obtained from the subject as having one or more gain-of-function mutations in the gene encoding PLCγ2.

13. The method of any one of claims 1-3, wherein the disease or disorder associated with the constitutively activated PLCγ2 is an immune system disorder.

14. The method of claim 13, wherein the immune system disorder is PLCγ2-associated antibody deficiency and immune dysregulation syndrome (PLAID), familial cold autoinflammatory syndrome (FCAS3), autoinflammation, antibody deficiency, and immune dysregulation syndrome (APLAID), common variable immunodeficiency (CVID).

15. The method of claim 13 or 14, wherein the method further comprises identifying an immune cell obtained from the subject as having the constitutively activated PLCγ2 relative to a cell obtained from a healthy subject.

16. The method of any one of claims 13-15, wherein the method further comprises identifying an immune cell obtained from the subject as having one or more gain-of-function mutations in the gene encoding PLCγ2.

17. The method of any one of claims 3-16, wherein the one or more gain-of-function mutations comprise one or more mutations at amino acids P139, T168, I169, D334, Y482, N571, P664, R665, S707, A708, S718, R742, L845, L848, D993, D1140, M1141, F1142, or D1144 of SEQ ID NO: 1 or a deletion of one or more amino acids of SEQ ID NO: 1.

18. The method of claim 17, wherein the one or more gain-of-function mutations comprise one or more of the following mutations: P139S, T168A, I169V, D3341, Y482H, N571S, P664S, R665W, S707Y, S707P, S707F, A708P, S718R, R742P, L845F, L845V, L848P, D993Y, D993H, D1140G, D1140Y, D1140N, D1140E, D1140V, M1141L, M1141R, M1141K, F1142L, D1144N, or D1144G of SEQ ID NO: 1, or a deletion of at least amino acids L845-L848 of SEQ ID NO: 1, or a deletion of one or more nucleotides in exons 19-22 of the gene encoding SEQ ID NO: 1.

19. The method of any one of claims 4-12, wherein the one or more gain-of-function mutations comprise one or more mutations in SEQ ID NO: 1, wherein the one or more mutations are chosen from: D334H, P664S, R665W, S707Y, S707P, S707F, A708P, R742P, L845F, L845V, D993Y, D993H, D1140G, D1140Y, D1140N, D1140E, D1140V, M1141R, M1141K, F1142L, D1144N, and D1144G, or a deletion of at least S707-A708 of SEQ ID NO: 1, or a deletion of one or more nucleotides in exon 20 of the gene encoding SEQ ID NO: 1.

20. The method of any one of claims 13-16, wherein the one or more gain-of-function mutations comprise one or more mutations in SEQ ID NO: 1, wherein the one or more mutations are chosen from: P139S, T168A, I169V, Y482H, N571S, S707Y, S707P, A708P, S718R, L848P, M1141L, and M1141K, or a deletion of at least amino acids L845-L848 of SEQ ID NO: 1, or a deletion of one or more nucleotides of exons 19-22 of the gene encoding SEQ ID NO: 1.

21. The method of any one of claims 3-16, wherein the one or more gain-of-function mutations are located within a regulatory domain and/or a calcium binding domain of PLCγ2.

22. The method of claim 21, wherein the one or more gain-of-function mutations located within the regulatory domain and/or the calcium binding domain of PLCγ2 comprise one or more mutations at amino acids Y482, N571, P664, R665, S707, A708, S718, R742, L845, L848, D1140, M1141, F1142, or D1144 of SEQ ID NO: 1.

23. The method of claim 22, wherein the one or more gain-of-function mutations located within the regulatory domain and/or the calcium binding domain of PLCγ2 comprise one or more of the following mutations: Y482H, N571S, P664S, R665W, S707Y, S707P, S707F, A708P, S718R, R742P, L845F, L845V, L848P, D993Y, D993H, D1140G, D1140Y, D1140N, D1140E, D1140V, M1141L, M1141R, M1141K, F1142L, D1144N, or D1144G of SEQ ID NO: 1.

24. The method of any one of claims 1-23, wherein the BTK reducing molecule is a BTK degrader molecule.

25. The method of claim 24, wherein the BTK degrader molecule is a compound of Formula I: 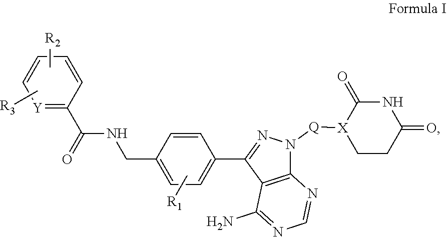
wherein X is CH or N;
wherein Y is CH or N;
wherein R₁ is chosen from H, C₁-C₃ alkyl, C₁-C₃ haloalkyl, dialkylamino group, amino group, —CN, hydroxyl, C₁-C₄ alkoxy, and halogen;
wherein each R₂ and R³ is independently chosen from H, halogen, —CN, hydroxyl, dialkylamino group, C₁-C₅ alkyl, deuterated C₁-C₅ alkyl, C₁-C₅ alkoxy, deuterated C₁-C₅ alkoxy, and C₁-C₅ haloalkyl;
wherein Q is L-W₁ or L-W₂;
wherein L is a linker of 2 to 20 carbon atoms in length, wherein one or more carbon atoms are optionally and independently replaced by a group chosen from C(═O), O, N(R₆), S, S(O), SO₂, C(O)NH, C(O)NCH₃, C(O)NCH₂CH₃, C₂-alkenyl, C₂-alkynyl, cycloalkyl, heterocycloalkyl, heterocycle, aryl, or heteroaryl, wherein each are independently substituted with 0, 1, 2 or 3 R₇;
wherein W₁ is chosen from 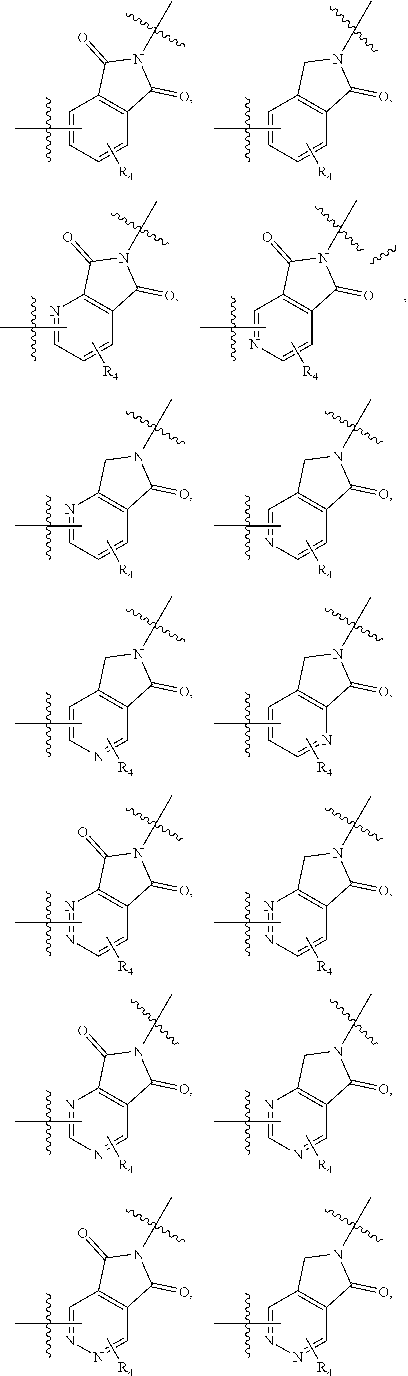 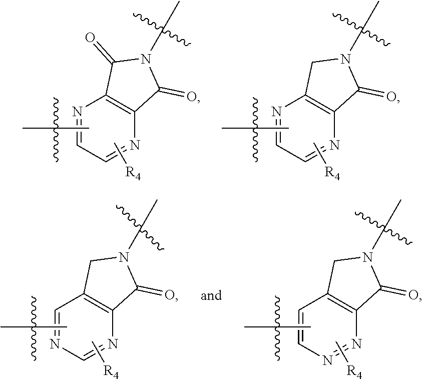
wherein R₄ is chosen from H, halogen, —CN, C₁-C₅ alkyl, C₁-C₅ alkoxy, and C₁-C₅ haloalkyl; and
wherein W₂ is 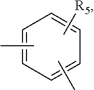
wherein R₅ is chosen from H, halogen, —CN, C₁-C₅ alkyl, deuterated C₁-C₅ alkyl, C₁-C₅ alkoxy, deuterated C₁-C₅ alkoxy, and C₁-C₅ haloalkyl;
wherein each R₆ is independently chosen from H, C₁-C₃ alkyl, —C(═O)—(C₁-C₃ alkyl), —C(═O)—O—(C₁-C₃ alkyl), and —C(═O)—NH—(C₁-C₃ alkyl), each of which is substituted with 0, 1, 2, or 3 R₇; and
wherein each R₇ is independently chosen from halogen, hydroxyl, amino group, C₁-C₃ alkyl, C₁-C₃ alkoxy, C₁-C₃ haloalkyl, —N(R₆)2, and —CN,
or a pharmaceutically acceptable salt thereof.

26. The method of claim 25, wherein L is 2 to 12 carbon atoms in length, wherein one or more carbon atoms are optionally and independently replaced by a group selected from C(═O), O, S, S(O), SO₂, C(O)NH, C(O)NCH₃, C(O)NCH₂CH₃, NH, NCH₃, NCH₂CH₃, C₂-alkynyl,

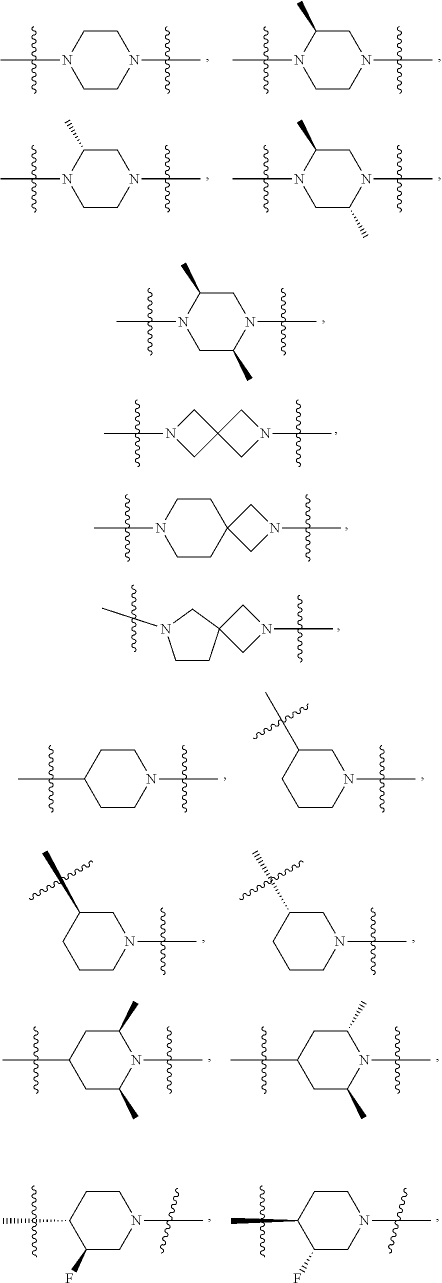

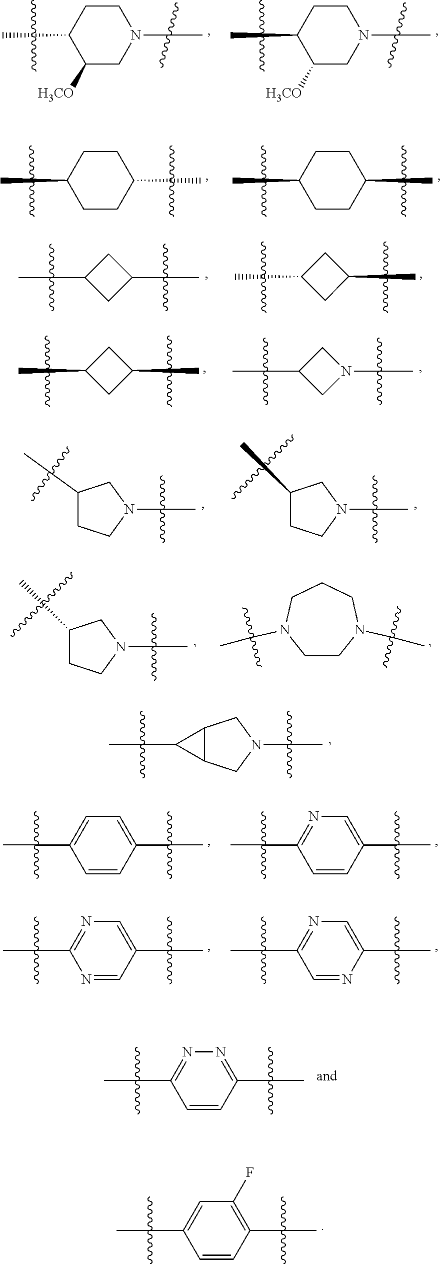

27. The method of claim 25 or 26, wherein the BTK degrader molecule is: 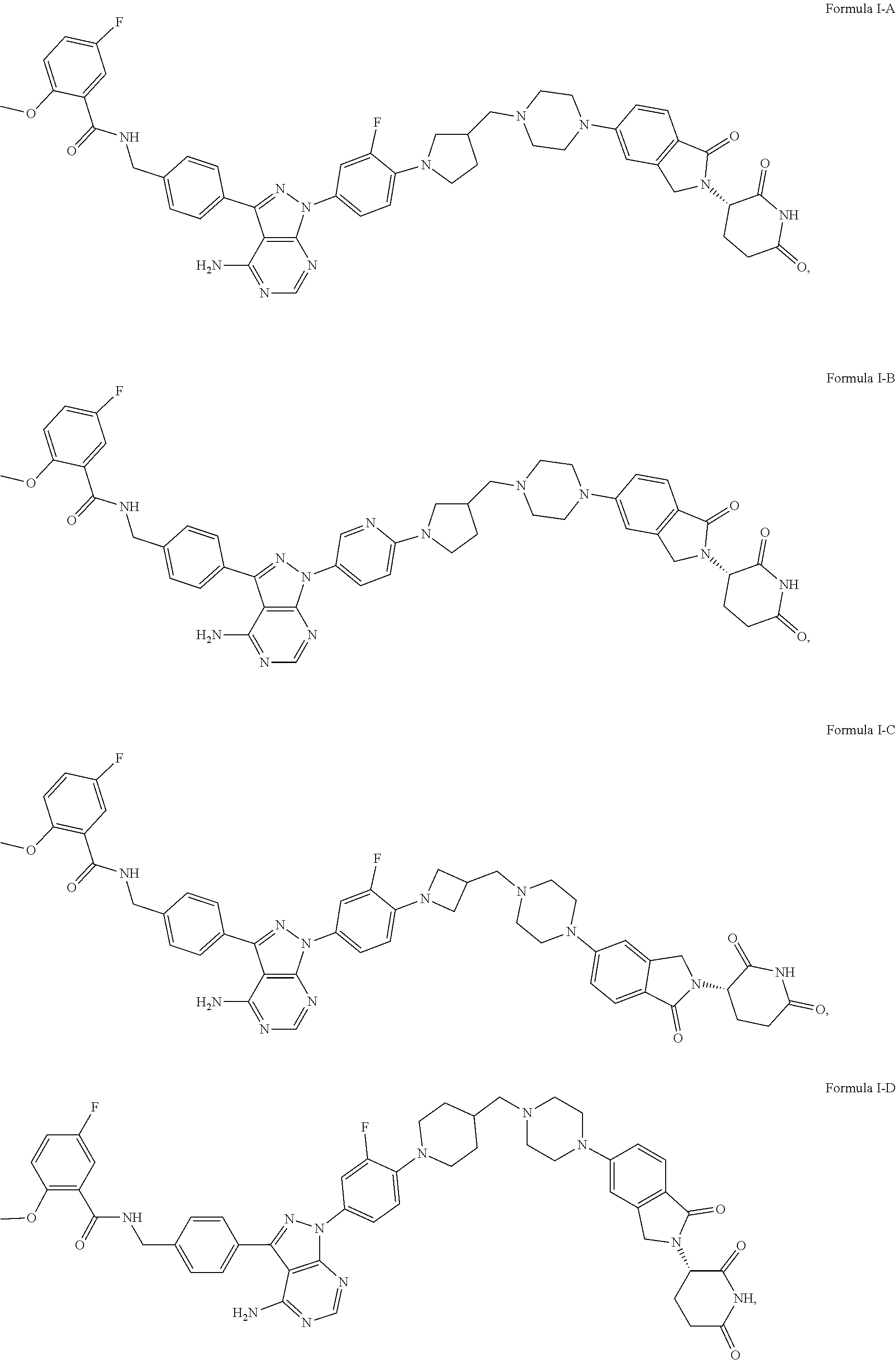 [SMILES: COc1ccc(F)cc1C(=O)NCc1ccc(-c2nn(-c3ccc(N4CCC(CN5CCN(c6ccc7c(c6)CN([C@H]6CCC(=O)NC6=O)C7=O)CC5)C4)c(F)c3)c3ncnc(N)c23)cc1 ; COc1ccc(F)cc1C(=O)NCc1ccc(-c2nn(-c3ccc(N4CCC(CN5CCN(c6ccc7c(c6)CN([C@H]6CCC(=O)NC6=O)C7=O)CC5)C4)nc3)c3ncnc(N)c23)cc1 ; COc1ccc(F)cc1C(=O)NCc1ccc(-c2nn(-c3ccc(N4CC(CN5CCN(c6ccc7c(c6)CN([C@H]6CCC(=O)NC6=O)C7=O)CC5)C4)c(F)c3)c3ncnc(N)c23)cc1 ; COc1ccc(F)cc1C(=O)NCc1ccc(-c2nn(-c3ccc(N4CCC(CN5CCN(c6ccc7c(c6)CN([C@H]6CCC(=O)NC6=O)C7=O)CC5)CC4)c(F)c3)c3ncnc(N)c23)cc1] 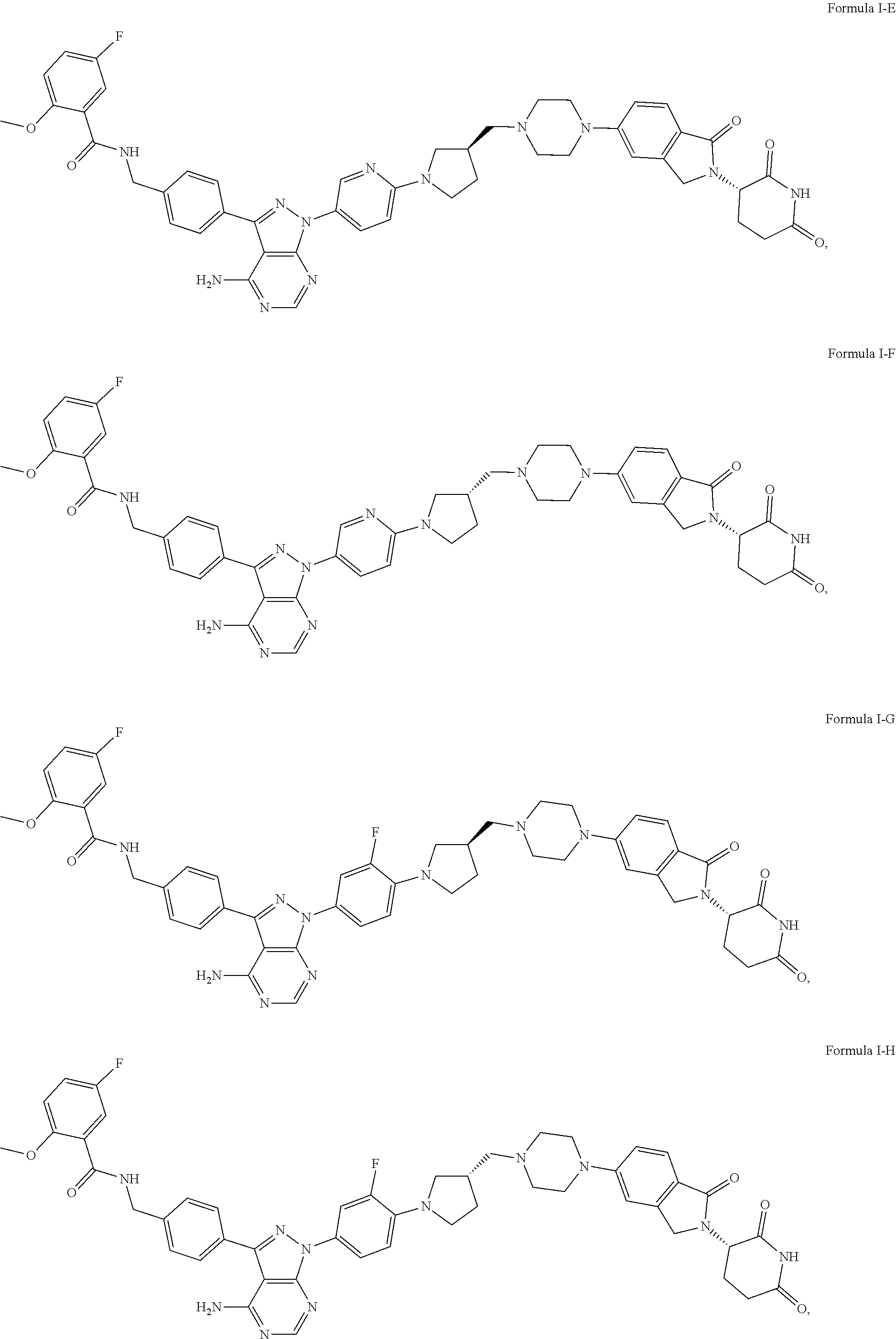 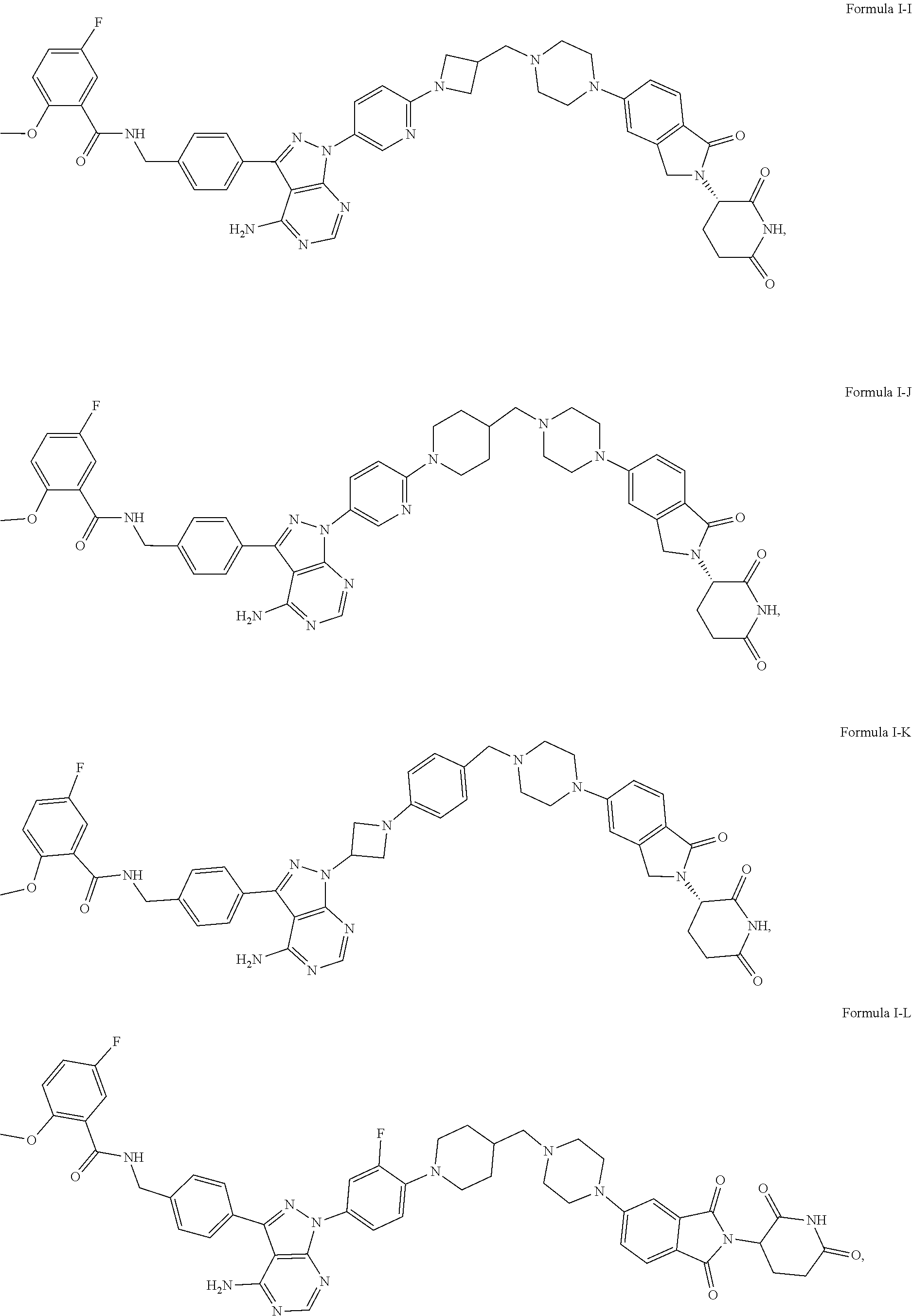 [SMILES: COc1ccc(F)cc1C(=O)NCc1ccc(-c2nn(-c3ccc(N4CC(CN5CCN(c6ccc7c(c6)CN([C@H]6CCC(=O)NC6=O)C7=O)CC5)C4)nc3)c3ncnc(N)c23)cc1 ; COc1ccc(F)cc1C(=O)NCc1ccc(-c2nn(-c3ccc(N4CCC(CN5CCN(c6ccc7c(c6)CN([C@H]6CCC(=O)NC6=O)C7=O)CC5)CC4)nc3)c3ncnc(N)c23)cc1 ; COc1ccc(F)cc1C(=O)NCc1ccc(-c2nn(C3CN(c4ccc(CN5CCN(c6ccc7c(c6)CN([C@H]6CCC(=O)NC6=O)C7=O)CC5)cc4)C3)c3ncnc(N)c23)cc1 ; COc1ccc(F)cc1C(=O)NCc1ccc(-c2nn(-c3ccc(N4CCC(CN5CCN(c6ccc7c(c6)C(=O)N(C6CCC(=O)NC6=O)C7=O)CC5)CC4)c(F)c3)c3ncnc(N)c23)cc1] 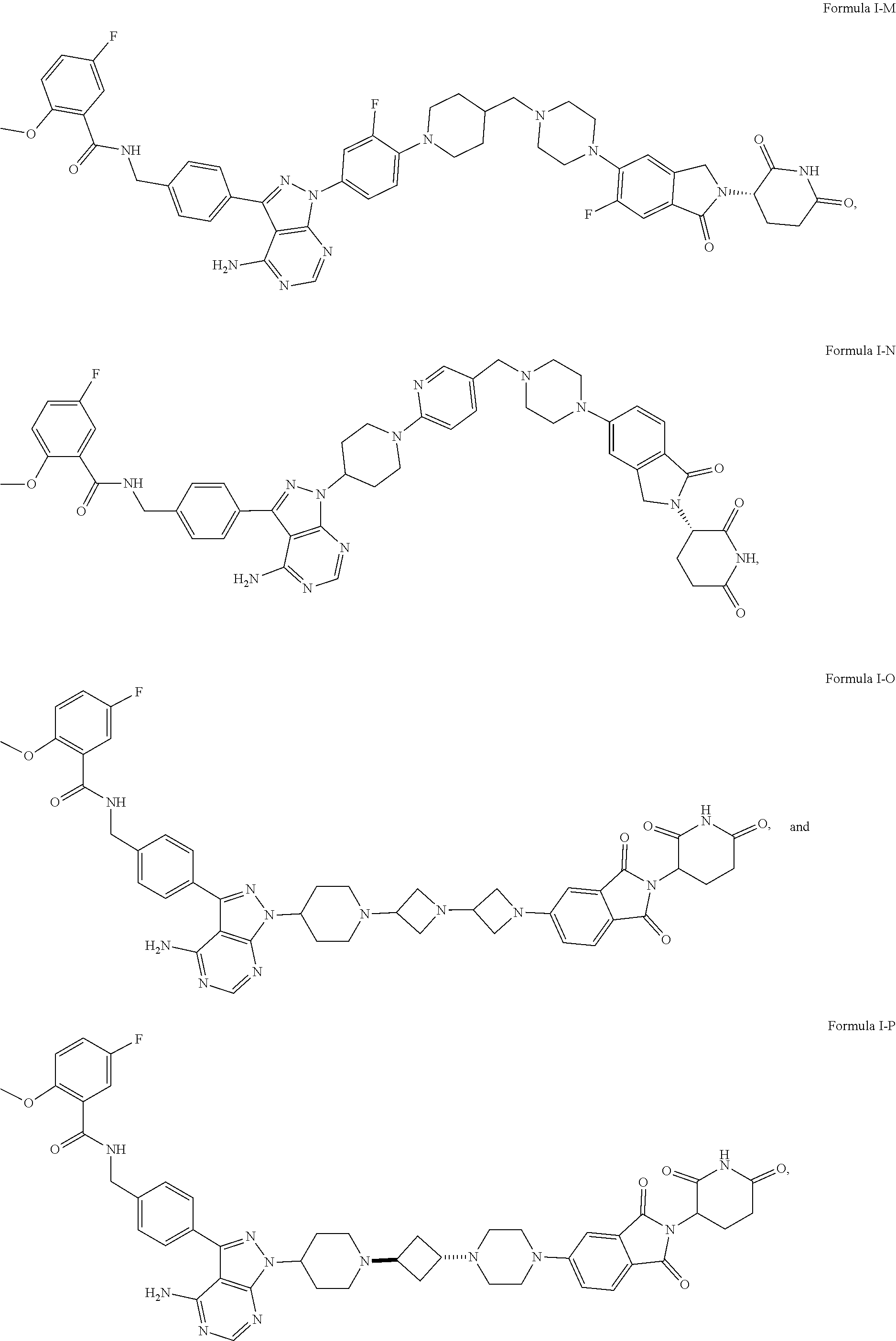 [SMILES: COc1ccc(F)cc1C(=O)NCc1ccc(-c2nn(-c3ccc(N4CCC(CN5CCN(c6cc7c(cc6F)C(=O)N([C@H]6CCC(=O)NC6=O)C7)CC5)CC4)c(F)c3)c3ncnc(N)c23)cc1 ; COc1ccc(F)cc1C(=O)NCc1ccc(-c2nn(C3CCN(c4ccc(CN5CCN(c6ccc7c(c6)CN([C@H]6CCC(=O)NC6=O)C7=O)CC5)cn4)CC3)c3ncnc(N)c23)cc1 ; COc1ccc(F)cc1C(=O)NCc1ccc(-c2nn(C3CCN(C4CN(C5CN(c6ccc7c(c6)C(=O)N(C6CCC(=O)NC6=O)C7=O)C5)C4)CC3)c3ncnc(N)c23)cc1 ; COc1ccc(F)cc1C(=O)NCc1ccc(-c2nn(C3CCN([C@H]4C[C@H](N5CCN(c6ccc7c(c6)C(=O)N(C6CCC(=O)NC6=O)C7=O)CC5)C4)CC3)c3ncnc(N)c23)cc1]
or a pharmaceutically acceptable salt thereof.

28. The method of any one of claims 24-27, wherein the BTK degrader molecule is administered to the subject at a dose of from about 0.1 mg/kg to about 500 mg/kg.

29. The method of any one of claims 1-23, wherein the BTK reducing molecule is a nucleic acid inhibitor molecule.

30. The method of claim 29, wherein the nucleic acid inhibitor molecule is an antisense oligonucleotide, a microRNA, a RNAi molecule, an aptamer, an antagomir, or a ribozyme.

31. The method of any one of claims 1-30, wherein the subject is human.
